(19) United States
(12) Patent Application Publication
(10) Pub. No.: US 2002/0115642 A1
(43) Pub. Date: Aug. 22, 2002

(54) Beta-lactam antibiotics

(76) Inventors: Ming Fai Chan, Encinitas, CA (US); Rosario S. Castillo, San Diego, CA (US); Qing Li, La Jolla, CA (US); Venkata Ramana Doppalapudi, San Diego, CA (US); Mark Stephen Hixon, San Diego, CA (US); Thomas J. Lobl, Foster City, CA (US)

Correspondence Address: BAKER & MCKENZIE, 660 HANSEN WAY, PALO ALTO, CA, 94304, US

(21) Appl. No.: 09/847,525

(22) Filed: May 1, 2001

Related U.S. Application Data
(60) Provisional application No. 60/201,642, filed on May 2, 2000.

(51) Int. Cl. A61K 31/675; A61K 31/545; A61K 31/546; C07D 501/14

(52) U.S. Cl. 514/79; 514/200; 514/202; 540/222; 540/229

(57) ABSTRACT

The present invention provides compositions comprising improved beta-lactam antibiotics and methods for applying these compositions to inhibit the growth of microbial infections. The improved antibiotics are capable of inhibiting the growth of both antibiotic sensitive and antibiotic resistant microorganisms In addition, the invention provides methods for treating a subject infected with a microorganism by administering the compositions of the invention.

### CROSS-REFERENCE TO RELATED APPLICATIONS

This application claims priority under 35 U.S.C. § 119(e) to U.S. Provisional Application No. 60/201,642, filed May 2, 2000, the contents of which are hereby incorporated by reference into the present disclosure.

### TECHNICAL FIELD

The present invention relates to the field of antibiotic therapy and specifically to compounds that inhibit the growth of infectious microorganisms. The present invention further relates to treatment of infections caused by antibiotic resistant microorganisms.

### BACKGROUND

Throughout this disclosure, various publications, patents and published patent specifications are referenced by bibliographic citation. The disclosures of these references are hereby incorporated by reference into the specification to more fully describe the state of the art to which this application pertains.

Antibiotics are drugs which have cytostatic or cytotoxic effects on target organisms. The key to success for an antibiotic is selectivity for the disease target, and lack of toxicity to the host, or patient. Many antibiotics are purified from cultures of microbial organisms themselves, while others are synthetic derivatives of naturally produced antibiotics year (Wilson, et al. in Harrison's Principles of Internal Medicine (12^{th} Ed). Publ: McGraw-Hill (1991). The most useful antibiotics against infections are those which attack a microbe-specific target. For example, β-lactam antibiotics interfere with cell wall synthesis by binding to cell wall precursors. Since mammalian cells lack the cell walls of bacteria, these drugs have a tremendous margin of safety for the patient.

Vancomycin, often called the antibiotic of last resort, inhibits synthesis and assembly of the second stage of cell wall peptidoglycan polymers by complexing with their D-alanyl-D-alanine precursor, which fits into a “pocket” in the vancomycin molecule, thereby preventing its binding to the peptidoglycan terminus that is the target of transglycolase and transpeptidase enzymes. In addition, vancomycin may impair RNA synthesis and injure protoplasts by altering the permeability of their cytoplasmic membrane.

Current agents have well characterized targets of action. Several examples are given below:

| Antibiotic Family | Example | Target |
|---|---|---|
| β-lactam antibiotics | Penicillins, cephalosporins | Cell wall biosynthesis |
| Sulfonamides | Sulfanilamide | Blocks synthesis of |
| | | tetrahydrofolate |
| Aminoglycosides | Streptomycin | Protein synthesis |
| Trimethoprim | — | Folate metabolism |
| Chloramphenicol | — | Protein synthesis |
| Vancomycin | — | Cell wall synthesis |

Other antibiotics work by blocking DNA replication, production of cellular RNA, or by modification of multiple cellular targets (Schaechter, et al. (1993) supra).

### Resistance Mechanisms

The widespread application of antimicrobial agents for the treatment of infectious diseases, particularly diseases caused by bacterial infections, has led to the development of antibiotic resistant strains of many microorganisms (Schaechter et al. (1993) in Mechanisms of Microbial Disease (2^{nd} Ed.) Publ. Williams & Wilkins p. 973; Murray (1997) Adv. Int. Med. 42:339-367). The problem has become so severe that the United States Center for Disease Control has advised the U.S. Senate to institute a nation-wide monitoring system to combat this threat (Smaglik (2000) Nature 407:437).

Resistance to antimicrobials was first recognized as penicillin resistance in staphylococci, and is now a recognized problem for the treatment of many bacterial infections, including essentially all nosocomial (hospital-acquired) bacterial infections (Murray (1997) supra). Nosocomial infections occur in 5% of patients admitted to the hospital (about 2 million patients per year in the United States); they cause an estimated 20,000 deaths per year, and contribute to an additional 60,000 hospital deaths. As early as 1991, it was estimated that nosocomial infections add about 7.5 million hospital days and $1 billion dollars in health care costs each year (Wilson, et al. in Harrison 's Principles of Internal Medicine (12^{th} Ed). Publ: McGraw-Hill (1991). The occurrence of resistance to antibiotics is now commonplace, and many of the mechanisms have been described (Schaechter, et al. (1993) supra; Murray (1997) supra). These mechanisms include overexpression of the target enzyme, expression of an antibiotic inactivating enzyme, or mutation of the target so that it is no longer recognized by the antibiotic. Examples are given below:

| Antibiotic | Principle mechanism of resistance |
|---|---|
| Penicillins and other β-lactam | Inactivation by β-lactamase |
| antibiotics | |
| Sulfanilamide | Mutation of dihydropteroate synthase |
| | target enzyme |
| Aminoglycosides | Inactivation by aminoglycoside |
| | modifying enzyme, or by target |
| | mutation |
| Trimethoprim | Mutation of dihydrofolate reductase |
| | target enzyme |
| Chloramphenicol | Inactivation by chloramphenicol |
| | transacetylase |
| Methicillin | Mutation of penicillin binding proteins |
| Vancomycin | Mutation in target cell wall peptide |

Although inactivation of antibiotics is probably the most common mechanism for drug resistance, resistance also occurs as a result of mutations in the drug targets themselves. The best characterized of these are mutations in the penicillin-binding-proteins (PBPs), leading to a decrease or loss in the binding.

Antibiotic resistant bacteria has increased as many organisms, e.g., Staphylococcus aureus, have developed resistance to several distinct antibiotics (the “multi-resistant” phenotype). The enzymes involved in drug resistance include the penicillinases, β-lactamases, cephalosporinases, and others. These enzymes inactivate antibiotics by modifying them to inactive compounds. Resistance caused by enzymes also includes antibiotic modification by choramphenicol acetyltransferases and other aminoglycoside modifying enzymes (Murray (1997) supra). Other mechanisms which contribute to antibiotic resistance include drug permeability mutations, expression of transport proteins that actively extrude antibiotics from target organisms, and mutations in the drug targets themselves (Murray (1997) supra).

The β-lactam antibiotics include penicillin, ampicillin, carbenicillin, and the cephalosporins (including cephalexin, cefaclor, cefoxitin, cefotaxime and cefoperazone). Because resistance is very common via production of high levels of β-lactamases, new drugs have been developed to inhibit these enzymes, thereby increasing the efficacy of the β-lactam antibiotics. Examples of β-lactamase inhibitors include clavulanate, timentin and sulbactam (Bush (1988) Clinical Microbial Rev. 1:109 123; Wilson, et al. (1991) supra; Schaechter, et al. (1993) supra). The combination of β-lactam antibiotic with β-lactamase inhibitor has extended the useful pharmacologic lifetimes of these antibiotics (Bush (1988) supra).

Vancomycin-resistant enterococci (VRE) emerged as important nosocomial pathogens in the United States. Strains of S. aureus that were intermediately resistant to vancomycin (VIRSA) were detected in the United States in 1997. VRE and VIRSA have raised serious concerns about the continued effectiveness of vancomycin in the treatment of these infections. Vancomycin-resistant enterococci produce two new enzymes, a ligase and a dehydrogenase, with formation of a new depsipeptide terminus D-ala-D-lactate, to the pentapeptide. This substitution allows cell wall synthesis to continue in the presence of the vancomycin.

Each “new” antibiotic derived from its previous generation (e.g., cephalosporin from penicillin) is been met with initial success, but is then replaced increasing reports of resistance. The progression of β-lactamases antibiotics is typical of the field. Each successive antibiotic is more resistant to degradation by β-lactamase, and the organism then produces larger amounts of the β-lactamase.

This is especially a problem for nosocomial infections (Wilson, et al. (1991) supra; Murray (1997) supra). The most common mechanism for transmission of the drug resistance phenotype is via plasmid, although some modulators of antibiotic resistance are located on the bacterial chromosome (Schaechter, et al. (1993) supra). The despair of the medical community has been addressed by the production of inhibitors of the β-lactamases. Unfortunately, although the β-lactamases have very much overlapping substrate specificities, they have evolved differently to have distinct, but related, amino acid sequences. This problem is expressed by the widely varying efficacy of each β-lactamase inhibitor for different enzymes. In addition, the new generation antibiotics are usually more toxic than their predecessors, and cannot be administered to patients in a convenient way. For example, vancomycin is used against organisms such as Streptococcus pneumonia and Enterococcus that are responsible for diseases such as pneumonia, meningitis, otitus media and many nosocomial infections, respectively. Novak et al. (Nature (1999) 399:590-593) recently reported the emergence of penicillin-resistant clinical isolates of S. pneumonia, which are also tolerant to a number of other antibiotics, including vancomycin.

Therefore, a need exists for a new generation of antibiotics that are not susceptible to the established drug-evasion mechanisms. This invention satisfies this need and provides related advantages as well.

### DISCLOSURE OF THE INVENTION

In one aspect, this invention provides prodrugs activated by a β-lactamase enzyme. The prodrugs selectively inhibiting the proliferation of microorganisms that expresses a β-lactamase enzyme as well as microorganisms that have become resistant or tolerant to conventional β-lactam antibiotics such as penicillin and vancomycin. The prodrug compounds of the invention will treat a subject infected with a microorganism that expresses a β-lactamase enzyme or/and a vancomycin-sensitive or resistant microbe. They also will treat a plant infested with a microbe expressing a β-lactamase enzyme or/and a vancomycin-sensitive or resistant microbe. The prodrug compounds of this invention are comprised of a β-lactam core covalently bound to a toxophore that is released by catalysis by a β-lactamase enzyme. They have the general structure shown below.

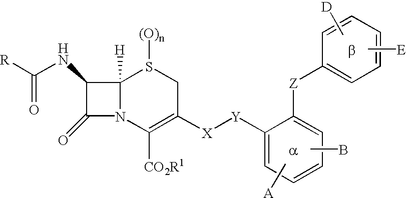

wherein n is 0, 1 or 2;

wherein A, B, D, and E are independently the same, different or absent and are selected from the group consisting of a halogen, H, CN, NO₂, CF₃, C(O)H, NH₂, N(R²)ₙ₁, and C(O)CH₃, OR², wherein R² is selected from the group consisting of H, lower alkyl, alkenyl group, and alkynyl group and wherein n1 is 0, 1 or 2;

wherein X is selected from the group consisting of CH₂, cis-CH═CH—CH₂—, trans-CH═CH—CH₂, —CH₂—O—C(O)—, —NH—C(O)—O—, —C≡C—CH₂, —PO₃—, —SO₃—, —SO₂, —NH—CH₂—CH₂—CH₂—NH—CO—, traceless Linker, and

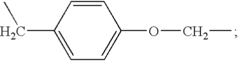

wherein Y is selected from the group consisting of —O—, —S—, and NR³, wherein R³ is selected from the group consisting of H, lower alkyl, alkenyl group, and alkynyl group;

wherein Z is selected from the group consisting of —O—, —C(O)—, —S—, α-C(O)—N(R⁴)-β, α-N(R⁴)—C(O)-β, and N(R⁴)ₙ₂, wherein R⁴ is selected from the group consisting of H. OH, R⁵, and OR⁵, wherein R⁵ is selected from the group consisting of H, lower alkyl, alkenyl group, and alkynyl group and wherein n2 is 0, 1 or 2;

wherein ring α connects Y to Z and is a benzene or a heterocycle selected from the group consisting of

wherein ring , connects to Z and is a benzene or a heterocycle selected from the group consisting of

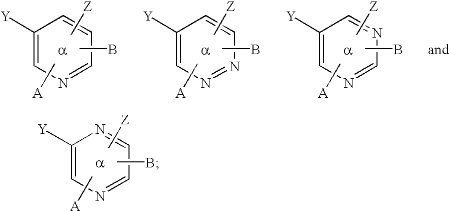

wherein ring β connects Y to Z and is a benzene or a heterocycle selected from the group consisting of

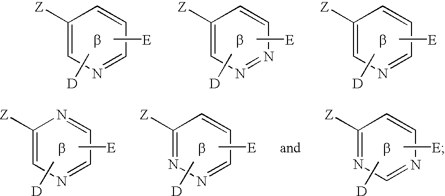

wherein R is selected from the group consisting of

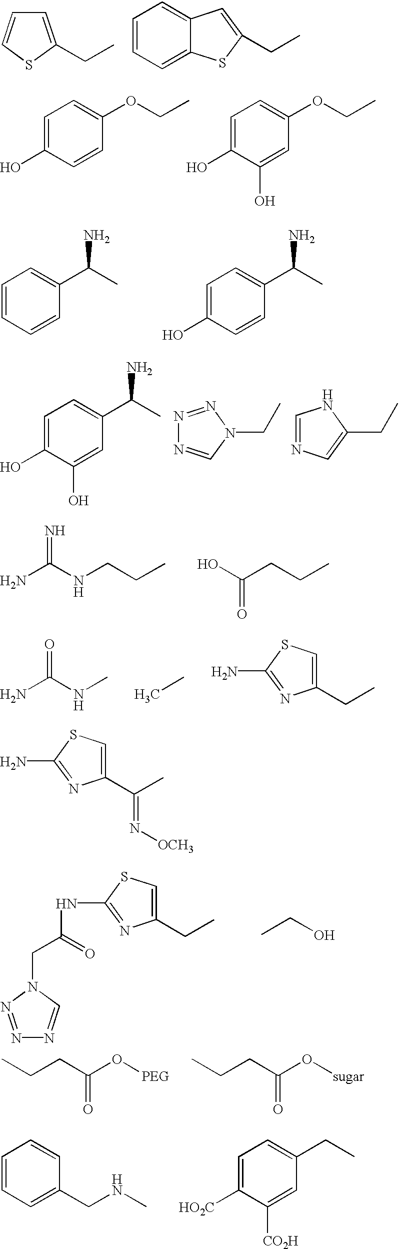

Ph-, PhCH₂— and PhOCH₂;

wherein R¹ is selected from the group consisting of H, Li, Na, sugar, THAM (2-amino-2-hydroxymethyl-1,3-propanediol), ammonium, methylamine, dimethyl amine, lower alkylamine, bis(lower alkyl)amine and polyethylene glycol (PEG);

and derivatives and pharmaceutically acceptable salts of the prodrug compounds.

Further provided by this invention is a process for preparing diphenylmethyl 3-(2-(2,4-dichlorophenoxy)_5-chlorophenoxy)methyl-7-β-(o-hydroxy)benzylidenamino-3-cephem-4-carboxylate.

### BRIEF DESCRIPTION OF THE FIGURES

FIG. 1 shows a method for synthesis of intermediate compounds of the invention.

FIG. 2 shows a method for the final step in the synthesis of 3-(2-(2,4-dichlorophenoxy)-5-chlorophenoxy)methyl-7-(2-thienylacetamido)-3-cephem-4-carboxylic acid (Compound 9).

FIG. 3 shows a method for the final step in the synthesis of 3-(2-(2,4-dichlorophenoxy)-5-chlorophenoxy)methyl-7-(2-thienylacetamido)-1-oxo-3-cephem-4-carboxylate (Compound 11).

FIG. 4 shows a method for synthesis of an intermediate of 3-((2-(2,4-dichlorophenoxy)-5-chlorophenoxy)carbonyloxy)methyl-7-(2-thienylacetamido)-2-cephem-4-carboxylic acid (Compound 15).

FIG. 5 shows a method for the final step in the synthesis of 3-((2-(2,4-dichlorophenoxy)-5-chlorophenoxy)carbonyloxy)methyl-7-(2-thienylacetamido)-2-cephem-4-carboxylic acid (Compound 15).

FIG. 6 shows a method for synthesis of 3-((2-(2,4-dichlorophenoxy)-5-chlorophenoxy)carbonyloxy)methyl-7-(2-thienylacetamido)-1-oxo-3-cephem-4-carboxylic acid (Compound 17).

FIG. 7 shows a method for synthesis of 3-(1-(2-(2,4-dichlorophenoxy)-5-chlorophenoxy)-3-propenyl)-7-(2-thienylacetamido)-3-cephem-4-carboxylic acid (Compound 24).

FIG. 8 shows a method for synthesis of 3-(2-(2,4-dichlorophenoxy)-5-chlorophenoxy)methyl-7-(1-tetrazoleacetamido)-3-cephem-4-carboxylic acid (Compound 29).

FIG. 9 shows a method for synthesis of 3-(2-(2,4-dichlorophenoxy)-5-chlorophenoxy)methyl-7-[2-(3H-imidazol-4-yl)]-acetamido-3-cephem-4-carboxylic acid (Compound 31).

FIG. 10 shows a method for synthesis of Compound (32) of the invention.

FIG. 11 shows a method for synthesis of 3-{3-[4-Chloro-2-(3,4-dichloro-phenylcarbamoyl)-phenoxy]-propenyl}-7-(2-thiophene-acetmido)-3-cephem-4-carboxylic acid (Compound 35).

FIG. 12 shows a method for synthesis of Compound (36) of the invention.

FIG. 13 shows a method for synthesis of Compounds 37 to 47 of the invention which includes: 3-(2-(2,4-dichlorophenoxy)-5-chlorophenoxy)methyl-7-(1-phenyl-2-aminoacetamido)-3-cephem-4-carboxylic acid (Compound 37); 3-(2-(2,4-dichlorophenoxy)-5-chlorophenoxy)methyl-7-(1-phenyl-2-aminoacetamido)-3-cephem-4-carboxylic acid (Compound 38); 3-(2-(2,4-dichlorophenoxy)-5-chlorophenoxy)methyl-7-[4-(2-aminothiazole)-yl-2-acetamido]-3-cephem-4-carboxylic acid (Compound 39); 3-(2-(2,4-dichlorophenoxy)-5-chlorophenoxy)methyl-7-[2-(4-hydroxyphenoxy)acetamido]-3-cephem-4-carboxylic acid (Compound 40); 3-(2-(2,4-dichlorophenoxy)-5-chlorophenoxy)methyl-7-[2-amino-2-(4-hydroxy-phenyl)acetamido)-3-cephem-4-carboxylic acid (Compound 41); 3-(2-(2,4-dichlorophenoxy)-5-chlorophenoxy)methyl-7-(3-guanidinopropyl)acetamido-3-cephem-4-carboxylic acid (Compound 42); and 3-[5-chloro-2-(2,4-dichlorophenoxy)-phenoxymethyl]-7-{2-[2-(2-tetrazol-1-yl-acetamido)-thiazol-5-yl]-acetamido-3-cephem-4-carboxylic acid (Compound 43).

FIG. 14 shows the time profile of the hydrolysis of Compound (9) with release of triclosan catalyzed by TEM-1. Assays were conducted at 37° C. in 100 mM potassium phosphate buffer pH 7.2 with 1 mM EDTA and 0.5 μg/ml TEM-1 β-lactamase. Compound (9) catalysis was determined via fixed time incubations followed by acid quenching. Product formation (triclosan) was determined by integrated peak area of A260 nm following HPLC separation. Hydrolysis product (triclosan) was separated from Compound (9) by use of an HP1100 series HPLC equipped with an Alltech Adsorbosphere HS(C18)5u 150 mm×4.6 mm column. The mobile phase was isocratic containing 55% acetonitrile and 0.1% TFA producing retention times of 20.4 minutes and 24.1 minutes for triclosan and Compound (9), respectively. Quantitation of triclosan was based on the A260 integrated peak area as compared to triclosan standards. TEM-1 blank experiment showed that Compound (9) was stable up to 5 hours at 37° C.

FIG. 15 shows the release of triclosan from Compound (9) in vivo. E coli N and β-lactamase expressing E. coli/TEM-1 were treated with Compound (9) under the condition described in Example 15. Aliquots of culture were sampled at 0, 5 and 15 minutes. The amount of Compound (9) and triclosan were analyzed by HPLC.

FIG. 16 shows the bactericidal activities of Compound (9) against S. aureus (ATCC#700260). Compound (9) was added in log-phase S. aureus (ATCC#700260) and incubated at 37° C. The final concentration of cells at hour 0 was 1.3×10⁶ cells/ml, and Compound (9) was 0.6 μg/mL. After 4 and 24 hours incubation, the number of alive cells was calculated.

FIG. 17 shows the glucuronidation assay of Compound (9). Assays were conducted as outlined in Example 15, using 100 μM Compound (9), triclosan or cephalothin incubated with 100 μg human liver microsome at 37° C. for 1 hour. The reactants were analyzed on TLC plate, and glucuronidated compounds were visualized by MolecularDynamics Storm 820.

### MODES FOR CARRYING OUT THE INVENTION

As used herein, certain terms may have the following defined meanings.

The singular form “a,” “an” and “the” include plural references unless the context clearly dictates otherwise. For example, the term “a cell” includes a plurality of cells, including mixtures thereof.

The term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “lower alkyl, alkynyl, or alkenyl” means a straight, branched or cyclic group that is between one and ten carbons in length (a C₁-C₁₀), or alternatively a C₁-C₆, or alternatively a C₁-C₄-containing group.

The term “prodrug” means a precursor or derivative form of a pharmaceutically active agent or substance that is less cytotoxic to a target cell as compared to the drug metabolite. It is enzymatically activated or converted into the more active form.

A “composition” is intended to mean a combination of active agent and another compound or composition, inert or active. Examples of inactive carriers include but are not limited to a detectable agent or label and matrix for the controlled release of the prodrug. See U.S. Pat. No. 6,150,146.

A “pharmaceutical composition” is intended to include the combination of an active agent with a carrier, inert or active, making the composition suitable for diagnostic or therapeutic use in vitro, in vivo or ex vivo.

The term “effective amount” is to include therapeutically or prophylactically effective amounts. The term refers to an amount effective in treating or preventing an infection in a patient or an infestation in a plant either as monotherapy or in combination with other agents.

The term “prophylactically effective amount” refers to an amount effective in preventing infection in a subject or plant infestation.

A “subject” is a plant or a vertebrate such as a fish, an avian or a mammal, and preferably a human. Fish include, but are not limited to pets and farm animals. Avians include, but are not limited to pets, sport animals and farm animals. Mammals include, but are not limited to, murines, simians, humans, farm animals, sport animals, and pets.

The term “treating” refers to any of the following: the alleviation of symptoms of a particular disorder in a patient; the improvement of an ascertainable measurement associated with a particular disorder; or a reduction in microbial number. One of skill in the art can determine when a host has been “treated” by noting a reduction in microbial load or an alleviation in symptoms associated with infection.

The term “pharmaceutically acceptable carrier” and “biologically acceptable carrier” refer to a carrier or adjuvant that is administered to a host or patient, together with a compound of this invention, and which does not destroy the pharmacological activity thereof and is non-toxic, when administered in doses sufficient to deliver an effective amount of the compound. Examples of suitable carriers include liquid phase carriers, such as sterile or aqueous solutions, as well as those described below. Examples of pharmaceutically acceptable carrier include any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

The term “pharmaceutically acceptable salt, prodrug or derivative” relates to any pharmaceutically acceptable salt, ester, ether, salt of an ester, solvate, such as ethanolate, or other derivative of a compound of the present invention which, upon administration to a recipient, is capable of providing (directly or indirectly) a compound of this invention or an active metabolite or residue thereof. Specifically, in this invention, polyethylene glycol (PEG) is combined with the compounds of the invention as an ester of the CO₂R¹ position on the compound. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system).

Salts of the compounds of the present invention may be derived from inorganic or organic acids and bases. Examples of acids include hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts. Examples of bases include alkali metal (e.g., sodium) hydroxides, alkaline earth metal (e.g., magnesium) hydroxides, ammonia, compounds of formula NW₄⁺, wherein W is C₁₋₄ alkyl and THAM (2-amino-2-hydroxymethyl-1,3-propanediol).

Examples of salts include: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylproprionate, picrate, pivalate, propionate, succinate, tartrate, thiocyanate, tosylate and undecanoate. Other examples of salts include anions of the compounds of the present invention compounded with a suitable cation such as Na⁺, Li⁺, NH₄⁺, and NW₄⁺(wherein W is a C₁₋₄ alkyl group).

For therapeutic use, salts of the compounds of the present invention will be pharmaceutically acceptable. However, salts of acids and bases which are non-pharmaceutically acceptable may also find use, for example, in the preparation or purification of a pharmaceutically acceptable compound or for use to reduce microbial infestation in plants.

The term “traceless Linker” indicates a spacer or connector between two parts of a single molecule such that when a particular bond is severed between the two parts of the molecule, the connector which is still attached to the second part of the molecule, eliminates leaving no trace of itself. See, for example, F. M. H. de Groot et al. (2000) J. Med. Chem. 43:3093-3102.

A “control” is an alternative subject or sample used in an experiment for comparison purposes. A control can be “positive” or “negative”.

An “antibacterial agent” is a compound which is destructive to or prevents the growth of bacteria.

A “β-lactam resistant microorganism” is a microorganism with the ability to synthesize a protein that neutralizes a β-lactam antibiotic.

A “vancomycin resistant microorganism” is a microorganism with the mechanism of inhibiting cell wall biosynthesis which renders vancomycin ineffective against the microorganism.

“Antibiotic tolerant or sensitive microorganisms” will stop growing but do not die in the presence of the antibiotic. Antibiotic tolerance can be difficult to detect because conventional in vitro tests in general do not detect tolerant strains. The tolerant strains seem to be sensitive to the antibiotics. Novak, et al. (1999) supra.

“Inhibiting the growth” of a microorganism means reducing by contact with an agent, the rate of proliferation of such a microorganism, in comparison with a control microorganism of the same species not contacted with this agent.

The present invention provides compositions and methods for inhibiting the growth of infectious microorganisms that include β-lactam and vancomycin sensitive and resistant microorganisms. To date, it is known that the prodrugs compounds have two mechanisms of action. Because the prodrugs are activated by a β-lactamase enzyme, they are active against any microorganism that expresses this enzyme.

The invention further provides a means of taking advantage of a key disease resistance mechanism, the overproduction of β-lactamase enzyme, to modify these drugs locally, thus overcoming the resistance phenotype and selectively inhibiting the growth of the microbes.

They also inhibit the growth of microorganisms by inhibiting the activity of penicillin binding protein (PBP).

The compounds of the invention have the general structure shown below.

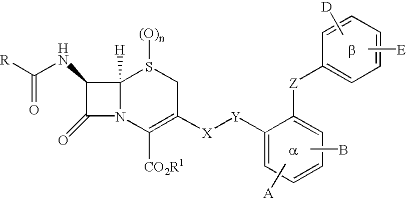

wherein n is 0, 1 or 2;

wherein A, B, D, and E are independently the same, different or absent and are selected from the group consisting of a halogen, H, CN, NO₂, CF₃, C(O)H, NH₂, N(R²)ₙ₁, and C(O)CH₃, OR², wherein R² is selected from the group consisting of H, lower alkyl, alkenyl group, and alkynyl group and wherein n1 is 0, 1 or 2;

wherein X is selected from the group consisting of CH₂, cis-CH═CH—CH₂—, trans-CH═CH—CH₂, —CH₂—O—C(O)—, —NH—C(O)—O—, —C≡C—CH₂, —PO₃—, —SO₃—, —SO₂, —NH—CH₂—CH₂—CH₂—NH—CO—, traceless Linker, and

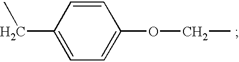

wherein Y is selected from the group consisting of —O—, —S—, and NR³, wherein R³ is selected from the group consisting of H, lower alkyl, alkenyl group, and alkynyl group;

wherein Z is selected from the group consisting of —O—, —C(O)—, —S—, α-C(O)—N(R⁴)-β, α-N(R⁴)—C(O)-β, and N(R⁴)ₙ₂, wherein R⁴ is selected from the group consisting of H, OH, R⁵, and OR⁵, wherein R⁵ is selected from the group consisting of H, lower alkyl, alkenyl group, and alkynyl group and wherein n2 is 0, 1 or 2;

wherein ring α connects Y to Z and is a benzene or a heterocycle selected from the group consisting of

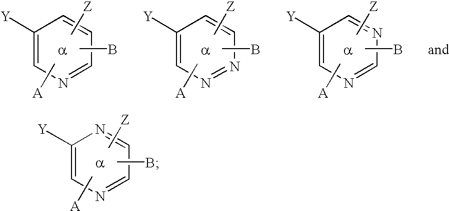

wherein ring β connects to Z and is a benzene or a heterocycle selected from the group consisting of

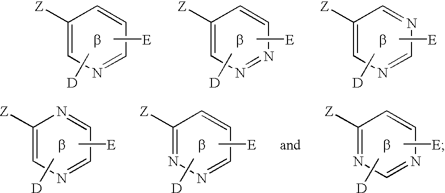

wherein R is selected from the group consisting of

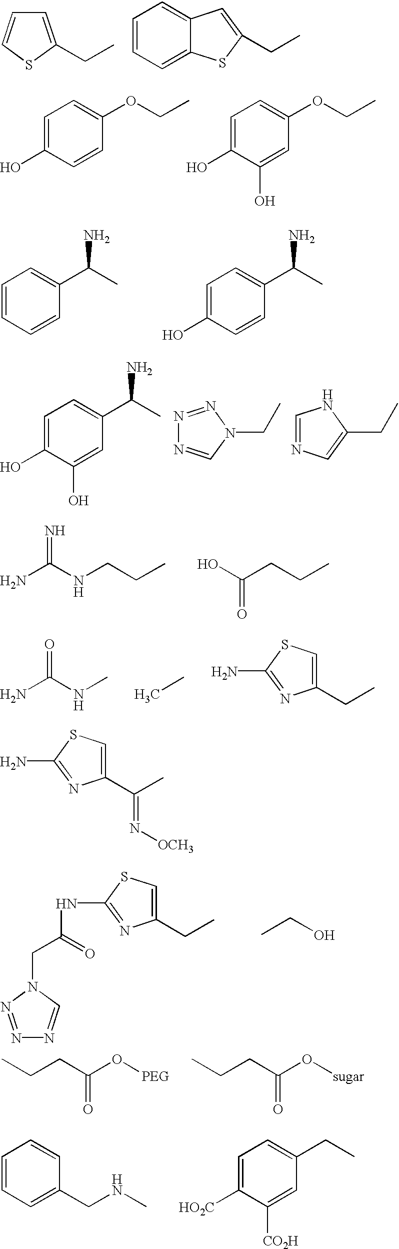

Ph-, PhCH₂— and PhOCH₂;

wherein R¹is selected from the group consisting of H, Li, Na, sugar, THAM (2-amino-2-hydroxymethyl-1,3-propanediol), ammonium, methylamine, dimethylamine, lower alkylamine, bis(lower alkyl)amine and polyethylene glycol (PEG); and derivatives and pharmaceutically acceptable salts of the prodrug compounds.

As used herein, the term “sugar” intends a chemical entity selected from the group consisting of sugar groups, thio-sugar groups, carbocyclic groups, and derivatives thereof. Examples of sugar groups include, but are not limted to, monosaccharide cyclic sugar groups such as those derived from oxetanes (4-membered ring sugars), furanoses (5-membered ring sugars), and pyranoses (6-membered ring sugars). Examples of furanoses include threo-furanosyl (from threose, a four-carbon sugar); erythro-furanosyl (from erythrose, a four-carbon sugar); ribo-furanosyl (from ribose, a five-carbon sugar); ara-furanosyl (also often referred to as arabino-furanosyl; from arabinose, a five-carbon sugar); xylo-furanosyl (from xylose, a five-carbon sugar); and lyxo-furanosyl (from lyxose, a five-carbon sugar). Examples of sugar group derivatives include “deoxy” , “keto” , and “dehydro” derivatives as well as substituted derivatives. Examples of thio sugar groups include the sulfur analogs of the above sugar groups, in which the ring oxygen has been replaced with a sulfur atom. Examples of carbocyclic groups include C₄ carbocyclic groups, C₅ carbocyclic groups, and C₆ carbocyclic groups which may further have one or more substituents, such as —OH groups.

Specific embodiments of the compounds have the structures as shown below:

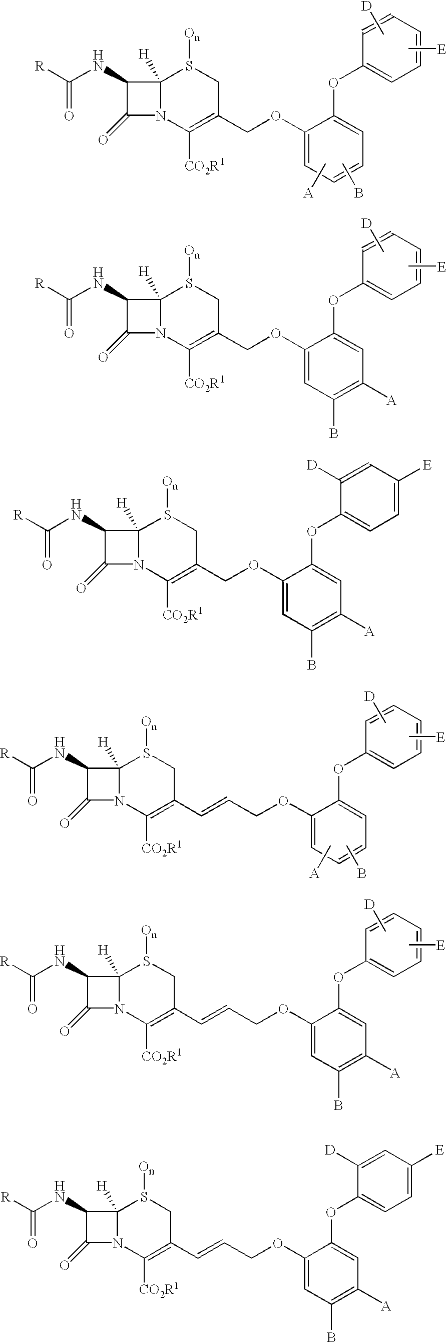

wherein n is 0, 1 or 2;

wherein A, B, D, and E are independently the same, different or absent and are selected from the group consisting of a halogen, H, CN, NO₂, CF₃, C(O)H, NH₂, N(R²)ₙ₁, and C(O)CH₃, OR², wherein R² is selected from the group consisting of H, lower alkyl, alkenyl group, and alkynyl group and wherein n1 is 0, 1 or 2;

wherein R is selected from the group consisting of

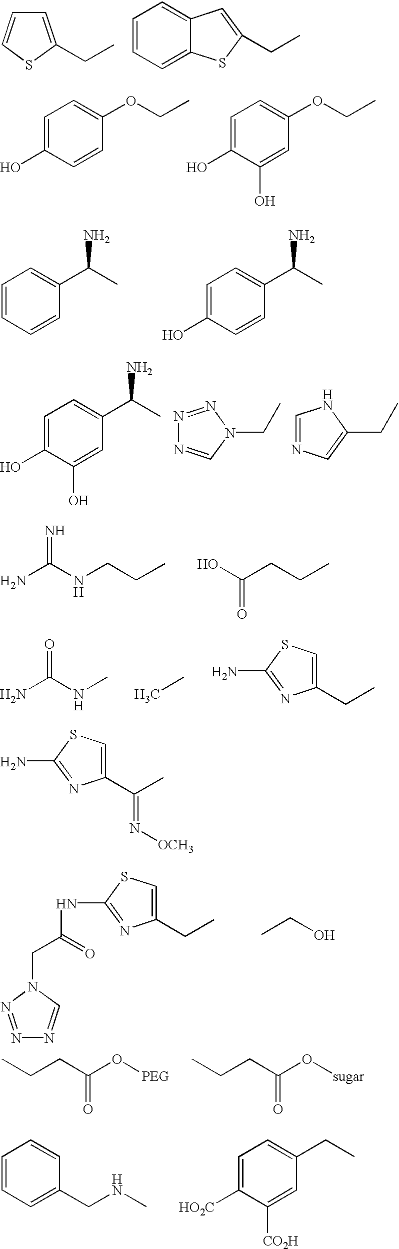

Ph-, PhCH₂— and PhOCH₂;

wherein R¹ is selected from the group consisting of H, Li, Na, sugar, THAM (2-amino-2-hydroxymethyl-1,3-propanediol), ammonium, methylamine, dimethylamine, lower alkylamine, bis(lower alkyl)amine and polyethylene glycol (PEG);

and derivatives and pharmaceutically acceptable salts of the prodrug compounds.

In a further aspect, this invention provides for the following specific compounds:

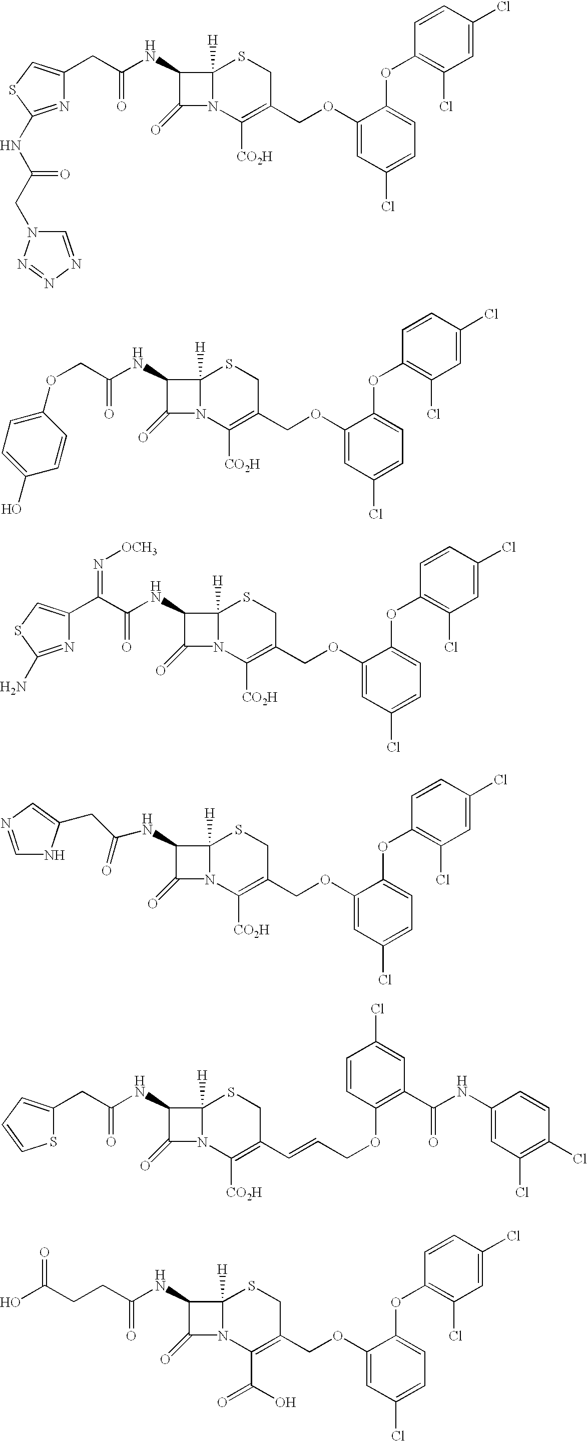

Also provided by this invention are compounds of this invention, combined with a carrier, inert or active. Examples include pharmaceutically, agriculturally or biologically acceptable carriers as defined above and those well known to one of skill in the art.

Further provided by this invention is a process for preparing diphenylmethyl 3-(2-(2,4-dichlorophenoxy)-5-chlorophenoxy)methyl-7-β-(o-hydroxy)benzylidenamino -3-cephem-4-carboxylate by reacting an effective amount of a compound having the structure (Compound 25):

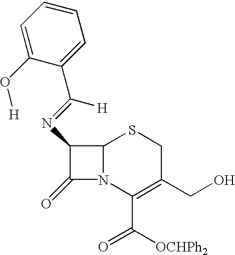

a) with an effective amount of triclosan and cooling to below 0° C.;

b) adding an effective amount of triphenylphosphine;

c) adding an effective amount of diisopropylazodicarboxylate and combining with an aqueous solvent and extracting with ethylacetate; and concentrating the compound, thereby preparing diphenylmethyl 3-(2-(2,4-dichlorophenoxy)-5-chlorophenoxy)methyl-7-β-(o-hydroxy)benzylidenamino -3-cephem-4-carboxylate.

In one aspect, the additions of steps a, b, or c, are added in a solution of anhydrous tetrahydrofuran (THF). In step a) the solution can be cooled to a temperature of less than −10° C., or less than −15° C., or alternatively, less than −20° C.

In another aspect, the method requires reacting a solution of compound 25 (1.0 mmol) and triclosan (1.25 mmol) in anhydrous THF (10 ml) and cooling to about −20° C. in a dry-ice bath under argon atmosphere. A solution of triphenylphosphine (1.25 mmol) in anhydrous THF (5.0 ml) is then added via a syringe. After 10 minutes, a solution of diisopropylazodicarboxylate (DIAD) (1.25 mmol) in anhydrous THF (5.0 ml) is slowly added using a syringe pump over 40 min. The reaction mixture is then poured into water (2×30 mL) and extracted with ethylacetate (2×25 mL). Combined ethylacetate extracts are washed with water, brine and dried over Na₂SO₄. Removal of volatiles followed by purification on silica gel column using 5% ethyl acetate in dichloromethane to provide diphenylmethyl 3-(2-(2,4-dichlorophenoxy)-5-chlorophenoxy)methyl-7-β-(o-hydroxy)benzylidenamino -3-cephem-4-carboxylate as light yellow solid.

The compounds of the invention are useful in methods to inhibit the growth of a microorganism that expresses or produces a β-lactamase enzyme or one that produces PBP. These methods require contacting the microorganism or cell infected with the microorganism with an effective amount of a compound of this invention.

The compounds of this invention also are useful to inhibit the proliferation of a microorganism that is either sensitive or resistant to a β-lactam antibiotic, e.g., penicillin or cephalosporin, or to vancomycin.

An aim of this invention is to provide compounds that can be modified by any β-lactamase to produce a secondary toxophore, thereby avoiding the problem of selecting the proper β-lactamase inhibitor. Because the β-lactam adduct of the compound will be broadly produced by β-lactamases of many species of bacteria (see, e.g., Vrudhula et al. (1995) J. Med. Chem. 38:1380-1385), a single compound will find utility for treating many different kinds of infections, previously resistant to treatment because of high levels of β-lactamase production by the target organism. This approach avoids the problem of mutation resistance encountered with β-lactamase inhibitors (Bush (1988) supra).

An alternative aim is to provide compounds that are toxic activity against non β-lactamase strains by the mechanism of inhibiting cell wall biosynthesis. When these compounds are treated with the bacterial strains that lack β-lactamase they inhibit penicillin binding protein (PBP), similar to conventional β-lactam antibiotics. Meanwhile, equal-molar of bactericide is formed, thus producing the bactericidal activity. Therefore, for β-lactamase negative microbes, the compounds of the present invention exert their antibacterial activity by formation of bactericidal agents and also by inhibition of PBP. For this reason, the compounds are especially useful against vancomycin-resistant, sensitive or tolerant microorganisms.

The compounds of the invention also selectively inhibit the growth of microorganisms that overexpress a β-lactamase enzyme. Overexpression can be caused by amplification of genes coding for the enzyme.

Amplification of genes associated with microbial resistance can be detected and monitored by a modified polymerase chain reaction (PCR) as described in Kashini-Sabet et al. (1988) Canc. Res. 48(20): 5775-5778 or U.S. Pat. No. 5,085,983. Alternative assays include enzyme activity assays (Miller (1992) A Laboratory Manual and Handbook for E. coli and Related Bacteria, Cold Spring Harbor Press) and via the polymerase chain reaction (Maher et al. (1995) Mol Cell Probes 9:265-276).

In one aspect, the compounds inhibit the growth of a bacteria by contacting the bacteria with an effective amount of the compound. The compounds are particularly suited to inhibit the growth of a β-lactam resistant or sensitive bacteria, e.g., a Gram-positive, Gram-negative bacterium, anaerobic bacterium or mycobacterium. Specific bacteria that can be inhibited include but are not limited to, a bacterium selected from the group consisting of: Staphylococcus aureus; Staphylococcus epidermidis and other coagulase-negative staphylococci; Streptooccus pyogenes; Streptococcus pneumoniae; Streptococcus agalactiae; Enterococcus species; Corynebacterium diphtheriae; Listeria monocytogenes; Bacillus anthracis; Neisseria meningitidis; Neisseria gonorrhoeae; Moraxella catarrhalis; Vibrio cholerae; Campylobacter jejuni; Enterobacteriaceae (includes: Escherichia, Salmonella, Klebsiella, Enterobacter); Pseudomonas aeruginosa; Acinetobacter species; Haemophilus influenzae; Clostridium tetani; Clostridium botulinum; Bacteroides species; Prevotella species; Porphyromonas species; Fusobacterium species; Mycobacterium tuberculosis; and Mycobacterium leprae. Examples of infections caused by these organisms are provided in Table 1, below.

The compounds are additionally effective against vancomycin sensitive or resistant bacteria. Specific bacteria that can be inhibited include, but are not limited to, a bacterium selected from the group consisting of vancomycin resistant Staphylococcus aureus, Staphylococcus epidermis, Enterococcus faecalis and Enterococcus faecium. Example of infections caused by these organisms are provided in Table 1, below.

TABLE 1

| | |
|---|---|
| Organism | Disease(s) |

GRAM-POSITIVE COCCI

| | |
|---|---|
| Staphylococcus aureus | Major human pathogen, bacteremia, pneumonia |
| Staphylococcus epidermidis and | Urinary tract infections, osteomyelitis, bacteremia |
| other coagulase-negative staphylococci | |
| Streptococcus pyogenes | Bacteremia, lymphangitis, pneumonia |
| Streptococcus pneumoniae | Pneumonia, otitis media, sinusitis |
| Streptococcus agalactiae | Primary bacteremia, pneumonia, endocarditis, |
| | osteomyelitis |
| Enterococcus species | Urinary tract infections, bacteremia, |
| | endocarditis, intra-abdominal and pelvic |
| | infections, neonatal sepsis |

GRAM-POSITIVE BACILLI

| | |
|---|---|
| Corynebacterium diphtheriae | Respiratory tract diphtheria, anterior nasal, |
| | faucial |
| Listeria monocytogenes | Bacteremia, meningoencephalitis |
| Bacillus anthracis | Acute infection |

GRAM-NEGATIVE COCCI

| | |
|---|---|
| Neisseria meningitidis | Endemic and epidemic disease |
| Neisseria gonorrhoeae | Genital infection, perihepatitis |
| Moraxella catarrhalis | Otitis media, lower respiratory tract |
| | infections, pneumonia, bacteremia |

GRAM-NEGATIVE BACILLI

| | |
|---|---|
| Vibrio cholerae | Feared epidemic diarrheal disease |
| Campylobacter jejuni | Acute enteritis, acute colitis, bacteremia |
| Enterobacteriaceae | Enteric infections, urinary tract infections, |
| (includes Escherichia, Salmonella, Shigella, | respiratory infections, bacteremia, |
| Klebsiella, Enterobacter) | bacillary dysentery |
| Pseudomonas aeruginosa | Endocarditis, respiratory infections, bacteremia, |
| | central nervous system infections |
| Acinetobacter species | Respiratory tract infections, bacteremia, |
| | genitourinary |
| Haemophilus influenzae | Pneumonia, meningitis, epiglottis, bacteremia |

ANAEROBIC BACTERIA

| | |
|---|---|
| Clostridium tetani | Tetanus |
| Clostridium botulinum | Botulism |
| Bacteroides species, | Postaspiration pleuropulmonary infection, |
| Prevotella species, Porphyromonas species | genital tract infection, intra-abdominal abcesses |
| and Fusobacterium species | |

MYCOBACTERIAL DISEASE

| | |
|---|---|
| Mycobacterium tuberculosis | Affects virtually every organ, most importantly |
| | the lungs |
| Mycobacterium leprae | Leprosy, Hansen's disease |

Contacting between the prodrug and the microorganism or cell infected with the microorganism can be achieved in vitro, ex vivo or in vivo. When the contacting is n vitro or ex vivo, the method is useful to inhibit the growth of microorganisms in solutions or on the surface by incorporation into an inert carrier. The methods of this invention can be practiced ex vivo using a modification of the method described in U.S. Pat. No. 5,399,346.

In one aspect, the invention provides a method for screening for an antimicrobial agent comprising contacting a sample containing a microbial cell with a test agent and contacting a second sample containing the microbial cell with a compound of this invention and comparing the ability of each to inhibit the growth of the microbe. The sample is intended to include microbial cells and subject cells infected with microorganisms that express β-lactamase or PBP. The test cells or tissue also are intended to include those that are infected with are resistant, tolerant or sensitive, e.g., to β-lactam or vancomycin. An infected cell can be a eucaryotic cell, i.e., a mammalian cell, e.g. a mouse cell, a rat cell, a hamster cell, or a human cell. The cell can be continuously cultured or isolated from an infected animal or human subject.

An example of a resistant cell suitable for use in the screen is TEM-52, an antibiotic-resitant E. coli which is reported to be more than 500-fold more resistant to the antiobiotic cefotaxime than wild-type E coli (Fletcher (2001) Nature Med. 19(3):217). In yet a further aspect, the bacterial cell is an antibiotic resistant bacterial cell. In a further aspect, at least one additional sample of cells is provided that is free of infection.

The compound is contacted with the sample under conditions that favor the activation of the compound by the β-lactamase enzyme and then assaying the sample for inhibition of microbial proliferation of the infected cells in the sample as compared to a control cell. Varying concentrations of the potential agent are contacted with the sample to determine the optimal effective concentration of the agent. Thus, in one aspect, this invention relates to the discovery and use thereof of agents that are selective substrates for enzymes that confer drug resistance to bacteria.

Also provided by this invention are kits containing the compounds as described herein and instructions necessary to perform the screen.

In vivo practice of the invention in an animal such as a rat or mouse provides a convenient animal model system that can be used prior to clinical testing of the therapeutic agent or compound. In this system, a potential compound will be successful if microbial load is reduced or the symptoms of the infection are ameliorated each as compared to an untreated, infected animal. It also can be useful to have a separate negative control group of cells or animals which has not been infected, which provides a basis for comparison.

When delivered to an animal, the following method is useful to further confirm toxicity of the prodrug. Groups of ICR-CD 1 male mice (˜22-25 g) are injected intraperitoneally, intravenously, intramuscularly, subcutaneously or are provided with oral dosages of various concentration of the ECTA compound. ECTA compound vehicle is used as control. Animals are observed twice a day for 14 days post-inoculation, and death is recorded.

The following method is used to determine the minimum lethal dose (MLD), median lethal dose (LD50) and the dose at which 100% of animals die (LD100) of the bacteria. Groups of ICR-CD1 male mice (˜22-25 g) are inoculated intraperitoneally with bacterial suspension between 10⁴ and 10⁸ CFU at half log steps and mucin 5% is used as a control. Animals are observed twice a day for 14 days post-inoculation, and death is recorded.

The following animal model shows the in vivo efficacy of ECTA compounds in mice. Mice are inoculated intraperitoneally with 0.5 ml of bacteria at 100 times the MLD. Mucin is used as control. A single or multiple injection of ECTA compound (intraperitoneal, intravenous, subcutaneous, intramuscular or oral) is administered post-inoculation. The ECTA compound vehicle is used as control. Animals are observed twice a day for 14 days post-inoculation, and death is recorded. The median effective dose (ED50) of ECTA compounds will be determined.

When practiced in vivo, the candidate compound is administered to the animal in effective amounts. As used herein, the term “administering” for in vivo and ex vivo purposes (if the target cell population is to be returned to the same (autologous) or another patient (allogeneic)) means providing the subject with an effective amount of the candidate prodrug effective to reduce bacterial load. In these instances, the agent or compound may be administered with a pharmaceutically acceptable carrier. The agents, compounds and compositions of the present invention can be used in the manufacture of medicaments and for the treatment of humans and other animals by administration in accordance with conventional procedures, such as an active ingredient in pharmaceutical compositions.

Methods of administering pharmaceutical compositions are well known to those of ordinary skill in the art and include, but are not limited to, microinjection, intravenous or parenteral administration. The compositions are intended for topical, oral, or local administration as well as intravenous, subcutaneous, or intramuscular administration. Administration can be effected continuously or intermittently throughout the course of the treatment. Methods of determining the most effective means and dosage of administration are well known to those of skill in the art and will vary with the prodrug used for therapy, the purpose of the therapy, the bacteria being treated, the severity of the infection, and the subject being treated. Single or multiple administrations can be carried out with the dose level and pattern being selected by the treating physician. For example, the compositions can be administered to a subject already suffering from an antibiotic resistant bacterial infection. In this situation, an effective “therapeutic amount” of the composition is administered to prevent continued, and to at least partially arrest, microbial growth and proliferation and ameliorate the symptoms associated with an infection.

However, the compounds can be administered to subjects or individuals susceptible to or at risk of developing an infection. In these embodiments, a “prophylactically effective amount” of the composition is administered to maintain cell viability and function at a level near to the pre-infection level.

It should be understood that by preventing or inhibiting unwanted infection in a subject, plant or individual, the compositions and methods of this invention also provide methods for treating, preventing or ameliorating the symptoms associated with a disease characterized by unwanted infection. Such diseases include but are not limited to bacterial infections, as shown in Table 1.

These methods can be further modified by co-administration or co-contacting with an additional known antibiotic or one that is yet to be discovered.

When an effective amount of a compound or composition of this invention is administered to a subject, such as an animal or human patient or applied to a plant, an infection or infestation is treated or prevented.

The compounds of this invention also can be employed in combination with other antibiotics for the inhibition of the replication or propagation of the bacteria and treatment of the associated conditions. Combination therapies, according to the present invention, comprise the administration of at least one compound of the present invention, and at least one other pharmaceutically or biologically active ingredient. The active ingredient(s) and pharmaceutically active agents may be administered simultaneously in either the same or different pharmaceutical formulations, or sequentially in any order. The amounts of the active ingredient(s) and pharmaceutically active agent(s), and the relative timings of administration will be selected in order to achieve the desired combined therapeutic effect. Preferably, the combination therapy involves the administration of one compound according to the invention and one of the agents mentioned herein below. The term “operative combination” is intended to include any chemically compatible combination of a compound of the present invention with other compounds of the present invention or other compounds outside the present invention, as long as the combination does not eliminate the anti-microbial activity of the compound of the present invention.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

More particularly, an agent of the present invention also referred to herein as the active ingredient, may be administered for therapy by any suitable route including oral, rectal, nasal, topical (including transdermal, aerosol, buccal and sublingual), vaginal, parental (including subcutaneous, intramuscular, intravenous and intradermal) and pulmonary. It will also be appreciated that the preferred route will vary with the condition and age of the recipient, and the disease being treated.

Ideally, the agent should be administered to achieve peak concentrations of the active compound at sites of disease. This may be achieved, for example, by the intravenous injection of the agent, optionally in saline, or orally administered, for example, as a tablet, capsule or syrup containing the active ingredient. Desirable blood levels of the agent may be maintained by a continuous infusion to provide a therapeutic amount of the active ingredient within disease tissue. The use of operative combinations is contemplated to provide therapeutic combinations requiring a lower total dosage of each component agent than may be required when each individual therapeutic compound or drug is used alone, thereby reducing adverse effects.

While it is possible for the agent to be administered alone, it is preferable to present it as a pharmaceutical formulation comprising at least one active ingredient, as defined above, together with one or more pharmaceutically acceptable carriers therefor and optionally other therapeutic agents. Each carrier must be “acceptable” in the sense of being compatible with the other ingredients of the formulation and not injurious to the patient.

Formulations include those suitable for oral, rectal, nasal, topical (including transdermal, buccal and sublingual), vaginal, parenteral (including subcutaneous, intramuscular, intravenous and intradermal) and pulmonary administration. The formulations may conveniently be presented in unit dosage form and may be prepared by any methods well known in the art of pharmacy. Such methods include the step of bringing into association the active ingredient with the carrier that constitutes one or more accessory ingredients. In general, the formulations are prepared by uniformly and intimately bringing into association the active ingredient with liquid carriers or finely divided solid carriers or both, and then if necessary shaping the product.

Formulations of the present invention suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient. This can be accomplished by using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention may be formulated as an ointment, cream, suspension, lotion, powder, solution, past, gel, spray, aerosol or oil. Alternatively, a formulation may comprise a patch or a dressing such as a bandage or adhesive plaster impregnated with active ingredients and, optionally, one or more excipients or diluents.

If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol or polyethylene glycol, and mixtures thereof. The topical formulations may desirably include a compound that enhances absorption or penetration of the agent through the skin or other affected areas. Examples of such dermal penetration enhancers include dimethylsulfoxide (DMSO) and related analogues.

The oily phase of the emulsions of this invention may be constituted from known ingredients in a known manner. While this phase may comprise merely an emulsifier (otherwise known as an emulgent), it desirably comprises a mixture of at lease one emulsifier with fat or oil or with fat and oil. Preferably, a hydrophilic emulsifier is included together with a lipophilic emulsifier that acts as a stabilizer. It is also preferred to include both oil and fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulfate.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing, in addition to the agent, such carriers as are known in the art to be appropriate.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the agent.

Formulations suitable for parenteral administration include aqueous and non-aqueous isotonic sterile injection solutions. These solutions may contain anti-oxidants, buffers, bacteriostats and solutes which render the formulation isotonic with the blood of the intended recipient and aqueous and non-aqueous sterile suspensions which may include suspending agents and thickening agents, and liposomes or other microparticulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above recited, or an appropriate fraction thereof, of a agent.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies.

These agents of this invention and the above noted compounds and their derivatives may be used for the preparation of medicaments for use in the methods described herein.

The clinical use of the prodrug antibiotics will likely follow well established guidelines. Dosage will likely be similar to those already employed for most other antibiotics. It is estimated that a dose of prodrug will be in the range of 100 mg to 1 gm, given once every eight hours, or once a day, for one or two weeks, or until the patient tests negative for infectious organisms.

The invention further encompasses a method of treating or protecting plants from microbial infections comprising applying an effective amount of the prodrug compound to the foliage roots or surrounding soil around the plants.

In order to achieve good dispersion and adhesion of the compounds as used to treat plants, it may be advantageous to formulate the compounds with components that aid dispersion and adhesion. Suitable formulations will be known to those skilled in the art.

This invention also provides a method for treating or protecting plants from infection by microorganism sensitive or resistant to β-lactam or vancomycin antibiotics by applying an effective amount of the compound to the foilage, roots or the soil surrounding the plants or roots. These isolated compounds can be combined with known pesticides or insecticides.

Compounds within the present invention when used to treat or protect plants from antibiotic resistant bacterial infections can be formulated as wettable powders, granules and the like, or can be microencapsulated in a suitable medium and the like. Examples of other formulations include, but are not limited to soluble powders, wettable granules, dry flowables, aqueous flowables, wettable dispersible granules, emulsifiable concentrates and aqueous suspensions. Other suitable formulations will be known to those skilled in the art.

This invention further provides a method for administering the prodrug compound to fish in an amount effective to either prevent or treat an antibiotic resistant bacterial infection. The compound may be administered by incorporating the compound into the food supply for the fish. Alternatively, the compound may be added to the water in which the fish live, or are contained within. Finally, the compound may be administered to the fish as a suitable pharmaceutical preparation. Other suitable formulations will be known to those skilled in the art.

Further provided is a process for producing compounds of this invention. See specific examples and figures, herein. The present invention further provides specific compounds representative of the general set of compounds. Thus, the present invention provides the following compounds:

3-(2-(2,4-dichlorophenoxy)-5-chlorophenoxy)methyl-7-(2-thienylacetamido)-3-cephem-4-carboxylic acid;

3-(2-(2,4-dichlorophenoxy)-5-chlorophenoxy)methyl-7-(2-thienylacetamido)-1-oxo-3-cephem-4-carboxylic acid;

3-(2-(2,4-dichlorophenoxy)-5-chlorophenoxy)methyl-7-(2-thienylacetamido)-1,1-dioxo-3-cephem-4-carboxylic acid;

3-(2-(2,4-dichlorophenoxy)-5-chlorophenoxy)methyl-7-(2-thienylacetamido)-2-cephem-4-carboxylic acid;

3-((2-(2,4-dichlorophenoxy)-5-chlorophenoxy)carbonyloxy)methyl-7-(2-thienylacetamido)-3-cephem-4-carboxylic acid;

3-((2-(2,4-dichlorophenoxy)-5-chlorophenoxy)carbonyloxy)methyl-7-(2-thienylacetamido)-1-oxo-3-cephem-4-carboxylic acid;

3-((2-(2,4-dichlorophenoxy)-5-chlorophenoxy)carbonyloxy)methyl-7-(2-thienylacetamido)-l ,1-dioxo-3-cephem-4-carboxylic acid;

3-((2-(2,4-dichlorophenoxy)-5-chlorophenoxy)carbonyloxy)methyl-7-(2-thienylacetamido)-2-cephem-4-carboxylic acid;

3-(1-(2-(2,4-dichlorophenoxy)-5-chlorophenoxy)-3-propenyl)-7-(2-thienylacetamido)-3-cephem-4-carboxylic acid;

3-(1-(2-(2,4-dichlorophenoxy)-5-chlorophenoxy)-3-propenyl)-7-(2-thienylacetamido)-1-oxo-3-cephem-4-carboxylic acid;

3-(1-(2-(2,4-dichlorophenoxy)-5-chlorophenoxy)-3-propenyl)-7-(2-thienylacetamido)-1,1-dioxo-3-cephem-4-carboxylic acid;

3-(1-(2-(2,4-dichlorophenoxy)-5-chlorophenoxy)-3-propenyl)-7-(2-thienylacetamido)-2-cephem-4-carboxylic acid;

3-(1-(2-(2,4-dichlorophenoxy)-5-chlorophenoxy)-3-propynyl)-7-(2-thienylacetamido)-3-cephem-4-carboxylic acid;

3-(1-(2-(2,4-dichlorophenoxy)-5-chlorophenoxy)-3-propynyl)-7-(2-thienylacetamido)-1-oxo-3-cephem-4-carboxylic acid;

3-(1-(2-(2,4-dichlorophenoxy)-5-chlorophenoxy)-3-propynyl)-7-(2-thienylacetamido)-1,1-dioxo-3-cephem-4-carboxylic acid;

3-(1-(2-(2,4-dichlorophenoxy)-5-chlorophenoxy)-3-propynyl)-7-(2-thienylacetamido)-2-cephem-4-carboxylic acid;

3-(2-(2,4-dichlorophenoxy)-5-chlorophenoxy)methyl-7-(1-tetrazoleacetamido)-3-cephem-4-carboxylic acid;

3-(2-(2,4-dichlorophenoxy)-5-chlorophenoxy)methyl-7-[2-(3H-imidazol-4-yl)]-acetamido-3-cephem-4-carboxylic acid;

3-{3-[4-chloro-2-(3,4-dichloro-phenylcarbamoyl)-phenoxy]-propenyl}-7-(2-thiophene-acetmido)-3-cephem-4-carboxylic acid;

3-(2-(2,4-dichlorophenoxy)-5-chlorophenoxy)methyl-7-phenylacetamido-3-cephem-4-carboxylic acid;

3-(2-(2,4-dichlorophenoxy)-5-chlorophenoxy)methyl-7-(1-phenyl-2-aminoacetamido)-3-cephem-4-carboxylic acid;

3-(2-(2,4-dichlorophenoxy)-5-chlorophenoxy)methyl-7-[4-(2-aminothiazole)-yl-2-acetamido]-3-cephem-4-carboxylic acid;

3-(2-(2,4-dichlorophenoxy)-5-chlorophenoxy)methyl-7-[2-(4-hydroxyphenoxy)acetamido]-3-cephem-4-carboxylic acid;

3-(2-(2,4-dichlorophenoxy)-5-chlorophenoxy)methyl-7-[2-Amino-2-(4-hydroxy-phenyl)-acetamido)-3-cephem-4-carboxylic acid;

3-(2-(2,4-dichlorophenoxy)-5-chlorophenoxy)methyl-7-(3-guanidinopropyl)acetamido-3-cephem-4-carboxylic acid;

3-[5-chloro-2-(2,4-dichlorophenoxy)-phenoxymethyl]-7-{2-[2-(2-tetrazol-1-yl-acetamido)-thiazol-5-yl]-acetamido-3-cephem-4-carboxylic acid;

3-(2-(2,4-dichlorophenoxy)-5-chlorophenoxy)methyl-7-(2-thienylacetamido)-1,1-dioxo-3-cephem-4-carboxylic acid;

3-(2-(2,4-dichlorophenoxy)-5-chlorophenoxy)methyl-7-[4-(2-aminothiazole)-yl-2-acetamido]-3-cephem-4-carboxylic acid;

3-((2-(2,4-dichlorophenoxy)-5-chlorophenoxy)carbonyloxy)methyl-7-(2-thienylacetamido)-1-oxo-3-cephem-4-carboxylic acid;

3-(2-(2,4-dichlorophenoxy)-5-chlorophenoxy)methyl-7-(1-tetrazoleacetamido)-1-oxo-3-cephem-4-carboxylic acid;

3-{3-[4-chloro-2-(2,4-dichloro-benzoyl)-phenoxy]-propenyl} -7-(2-thienylacetamido)-1-oxo-3-cepehm-4-carboxylic acid;

3-[4-chloro-2-(5-chloro-2-hydroxy-phenylsulfanyl)-phenoxymethyl]-7-(2-thienylacetamido)-3-cephem-4-carboxylic acid; and 3-(2-(2,4-dichlorophenoxy)-5-chlorophenoxy)methyl-7-(2-(3-nitrophenyl)acetamido)-3-cephem-4-carboxylic acid.

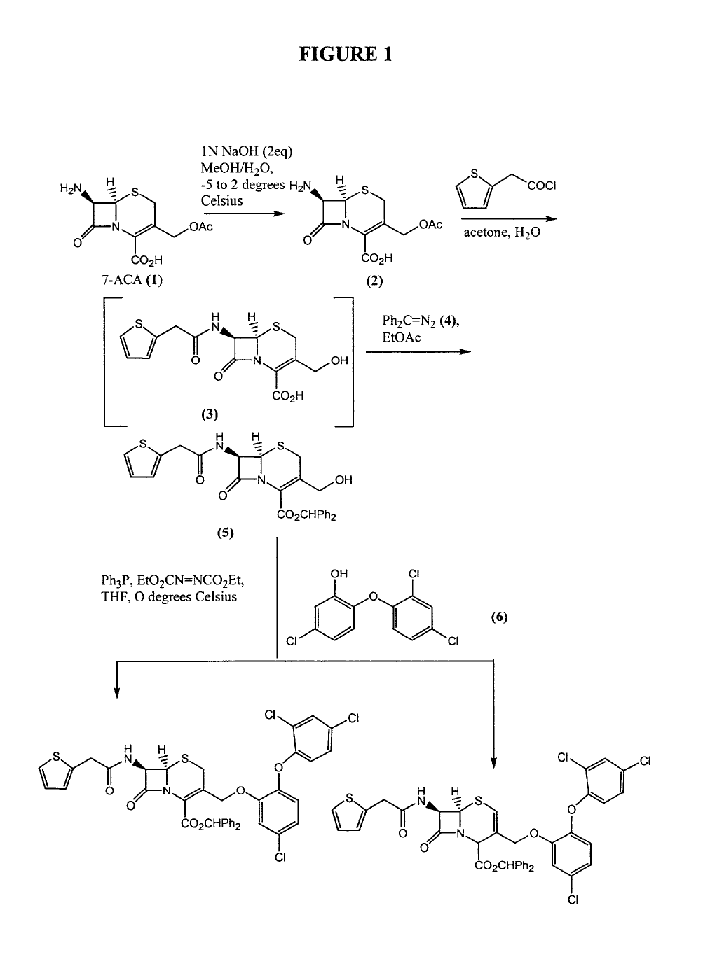

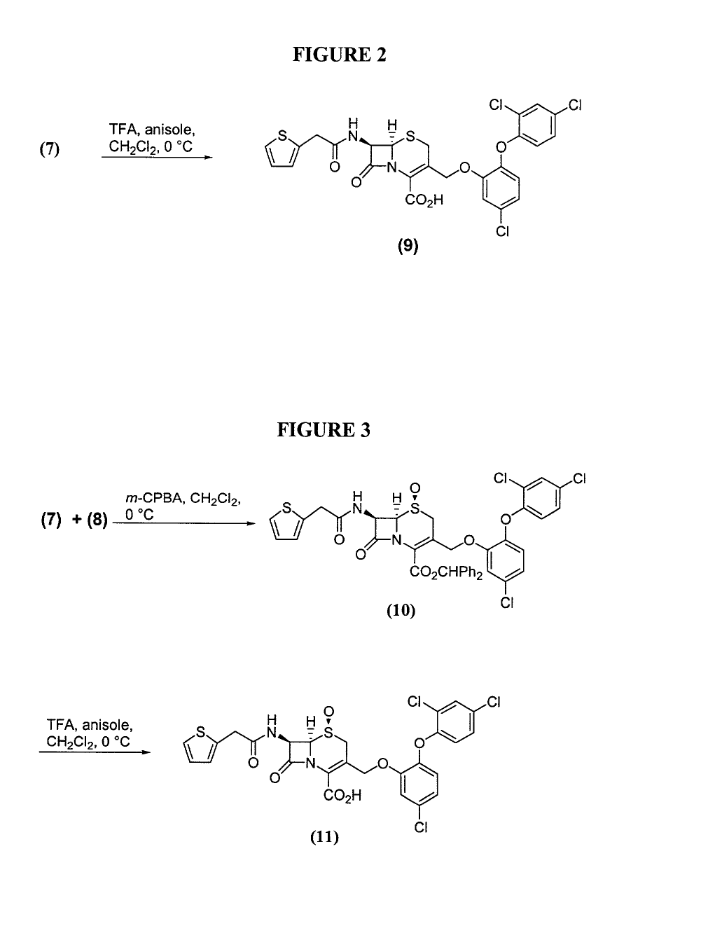

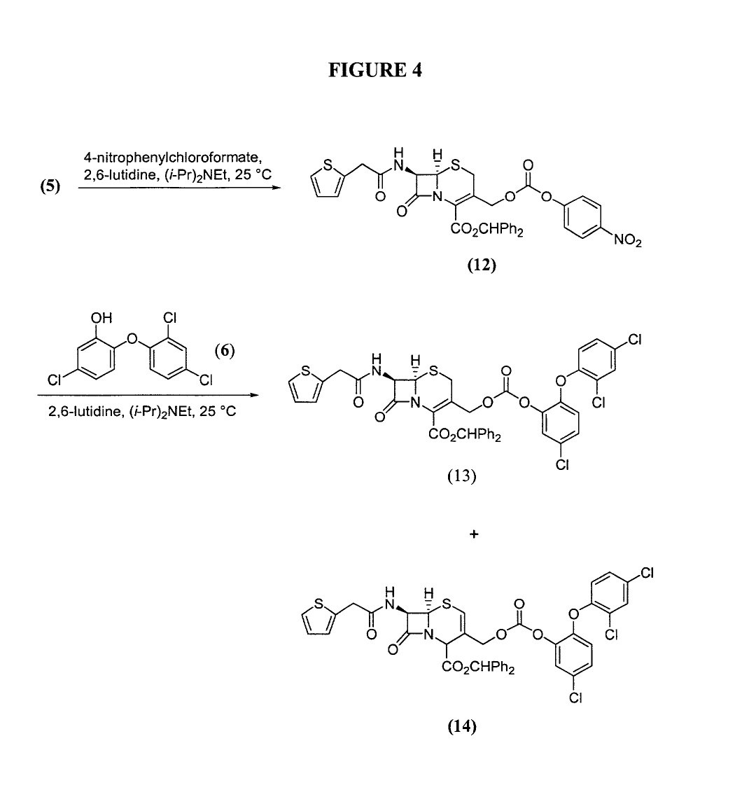

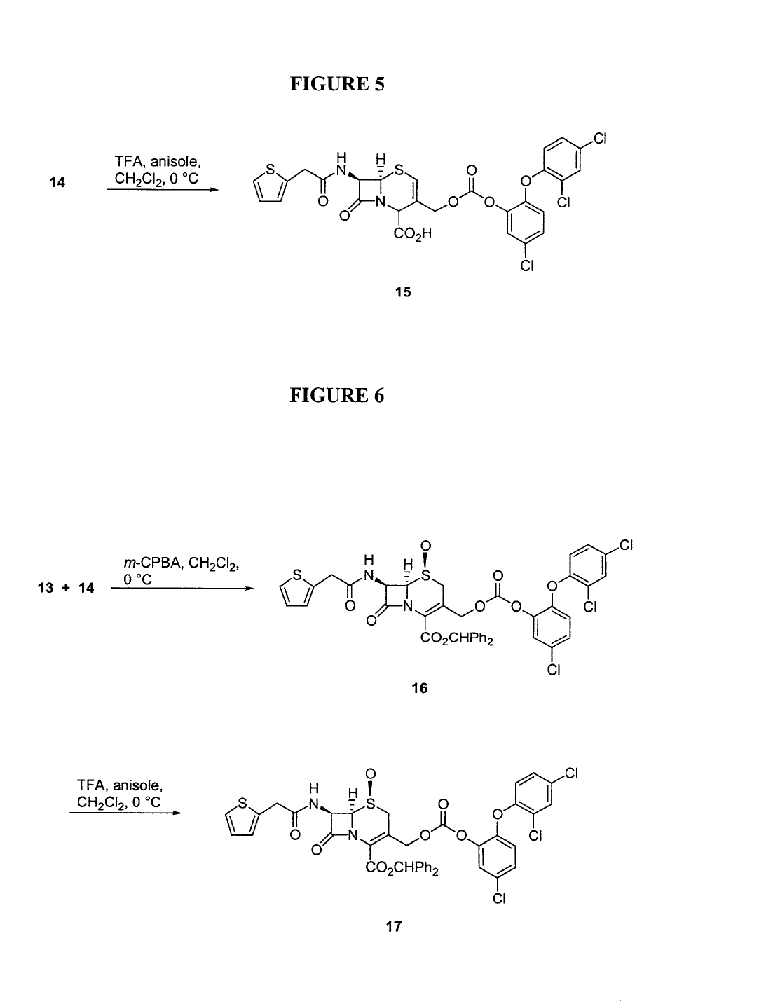

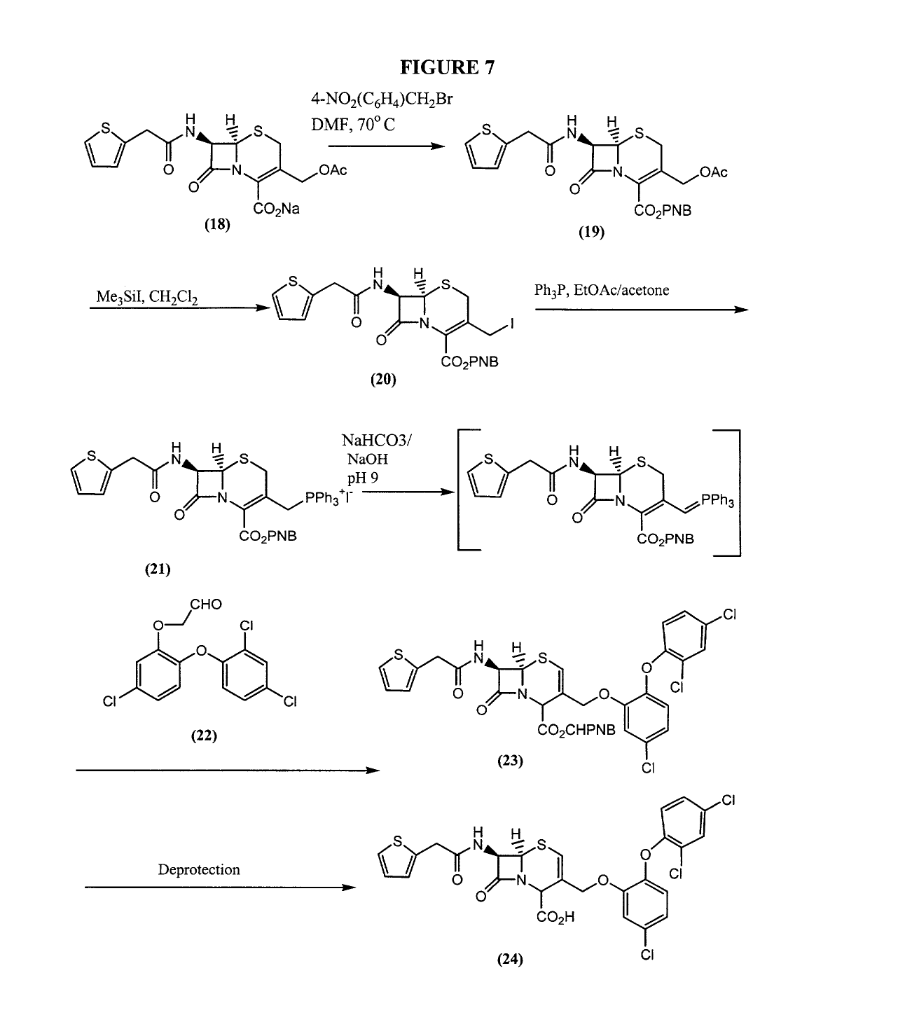

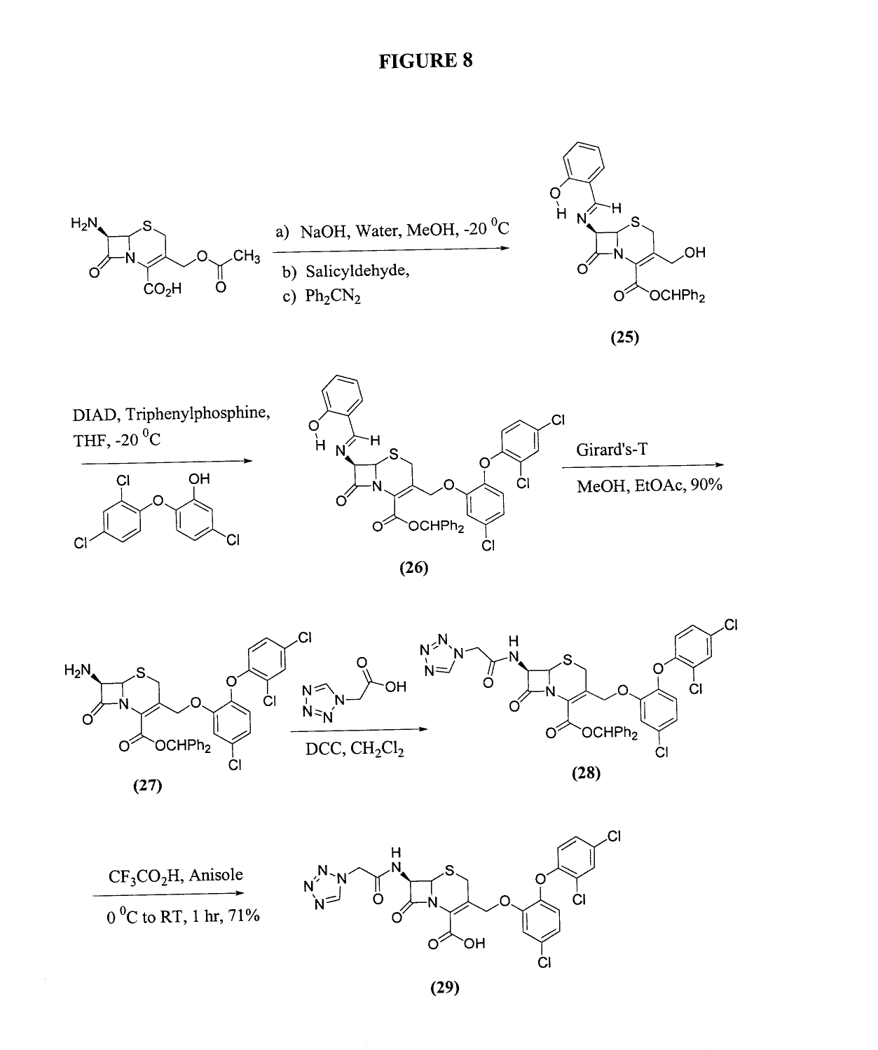

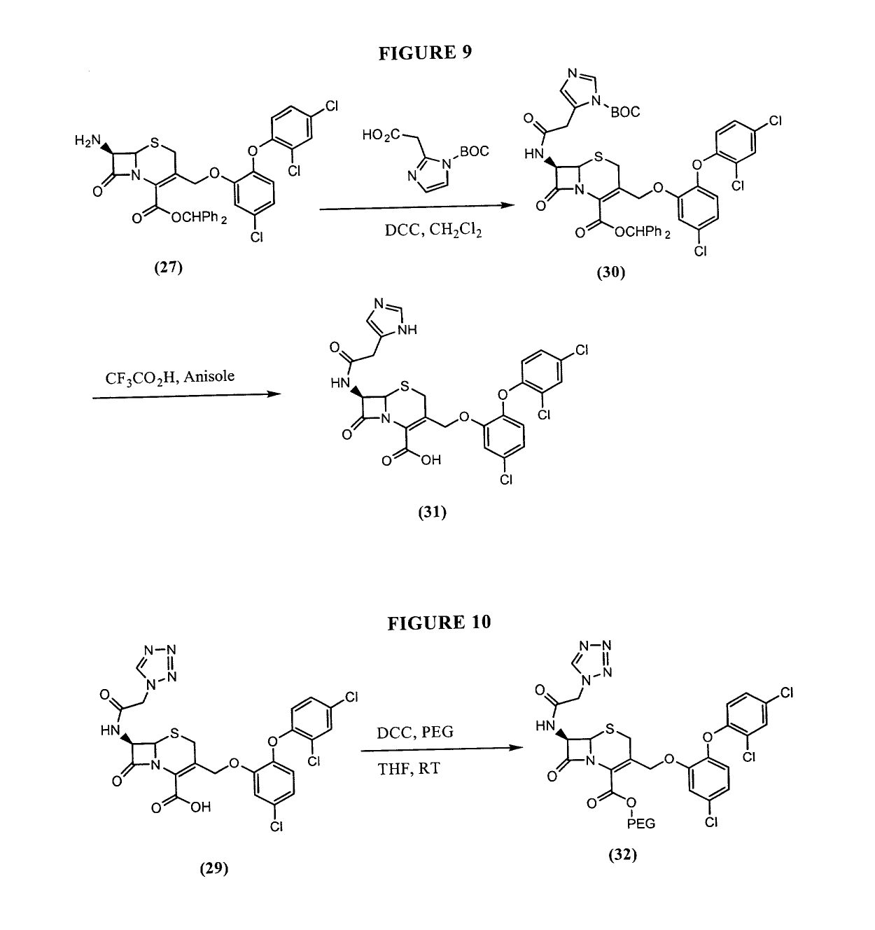

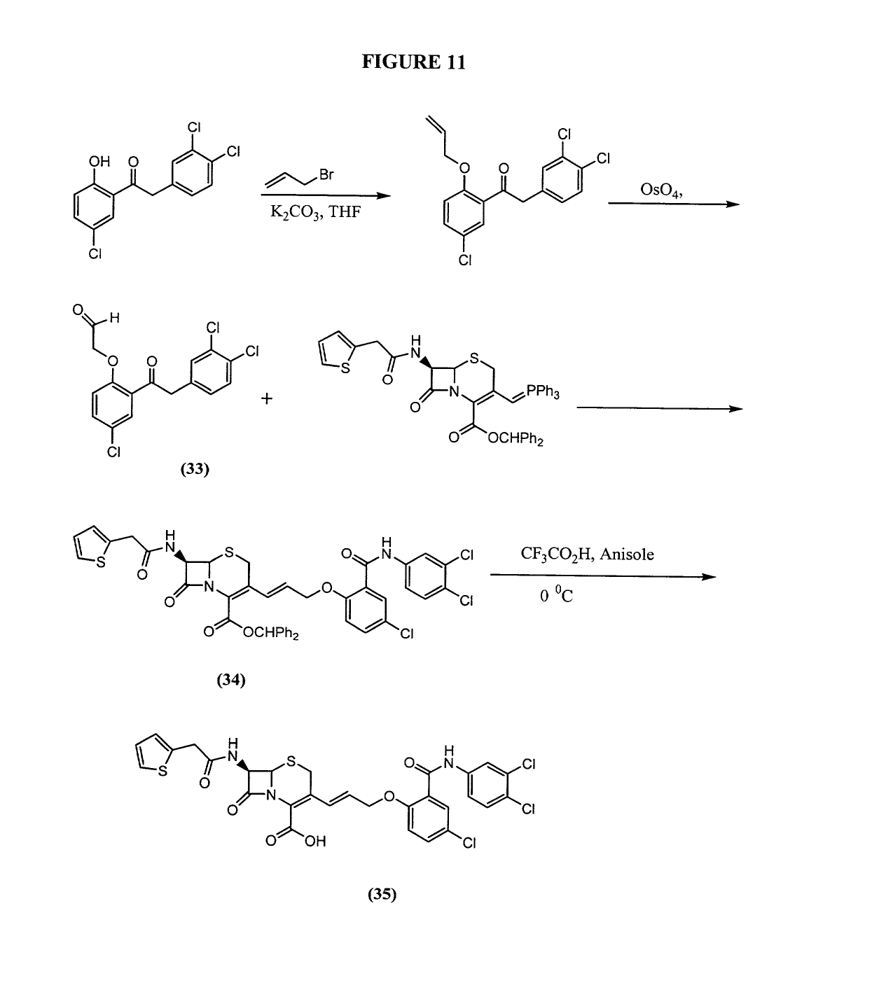

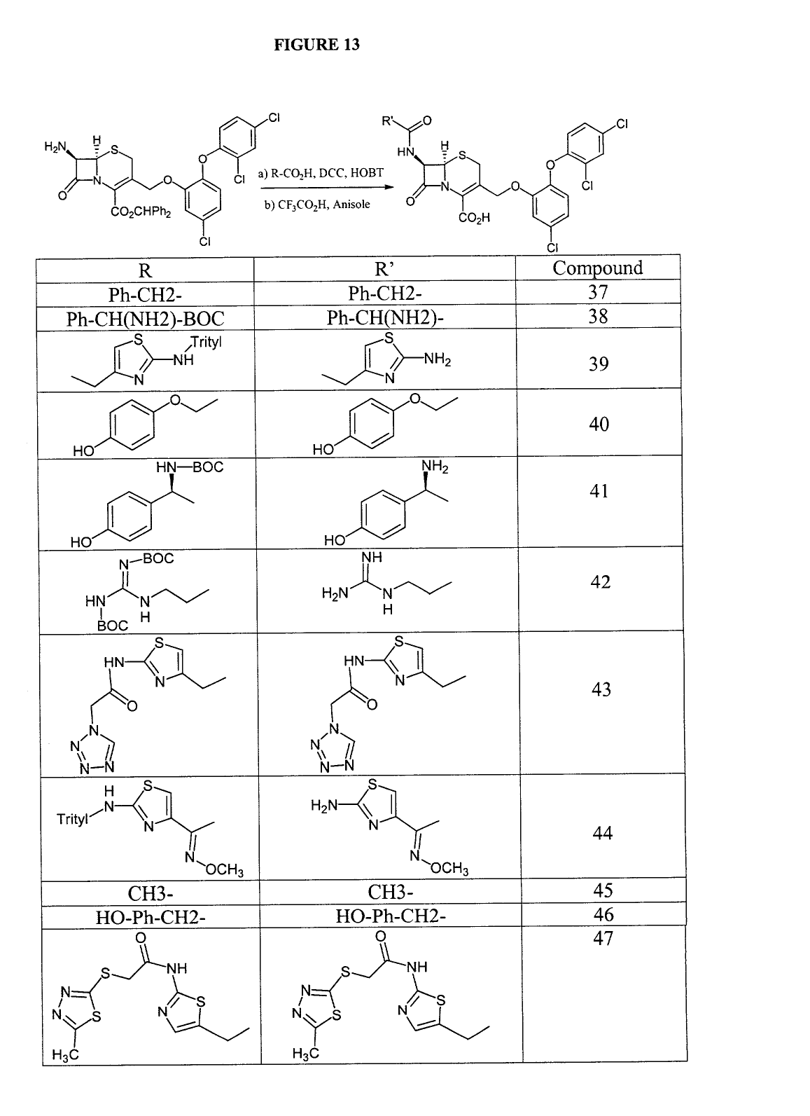

Details of methods for the synthesis of these and other related compounds are described in the examples below. Further descriptions of these methods are provided in FIGS. 1 through 13. The numbers of the compounds in the figure correspond to the numbers indicated in the text below.

The compounds of the present invention comprise alternative versions of the antibacterial toxophore triclosan or analogues of triclosan as the toxic compound that is released upon catalysis by β-lactamase. A number of alternative forms of the triclosan have been previously synthesized (For example, U.S. Pat. No. 3,993,779; U.S. Pat. No. 4,031,248).

While the present invention provides specific compounds and methods for the synthesis of such compounds, it will be apparent to individuals of skill in the art that alternative versions of such molecules can be prepared using well known methods. Thus, the present invention provides a series of related compounds as embodied in the general structure presented above.

### EXAMPLES

The following examples are intended to illustrate, not to limit the invention.

Chemical Synthesis

General synthesis schemes

The following general synthesis schemes A through H are used to produce the compounds of the present invention.

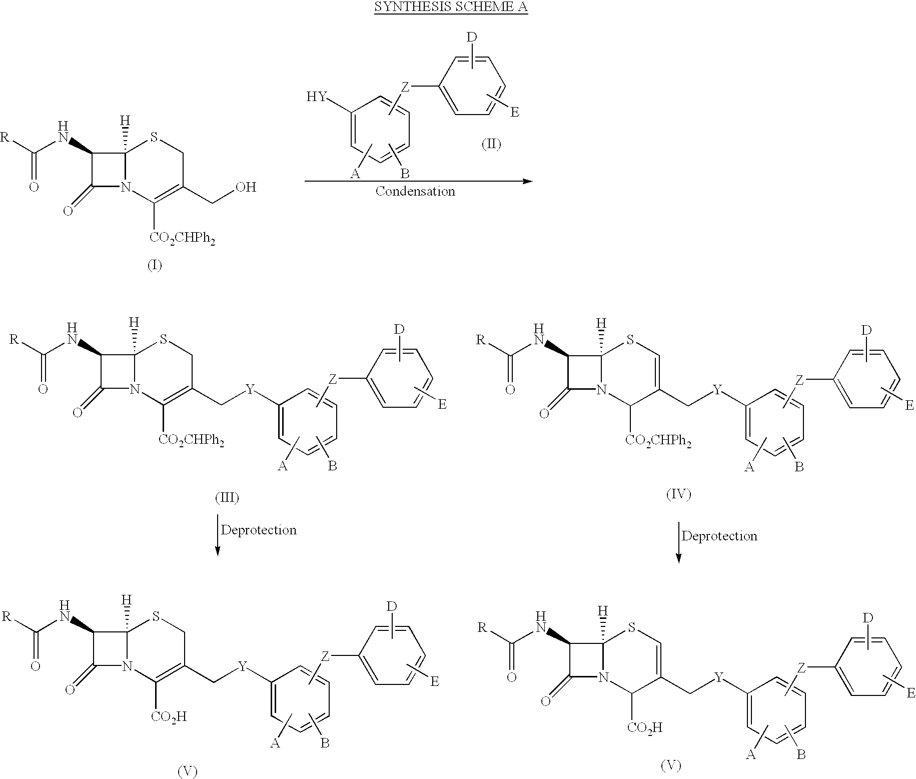

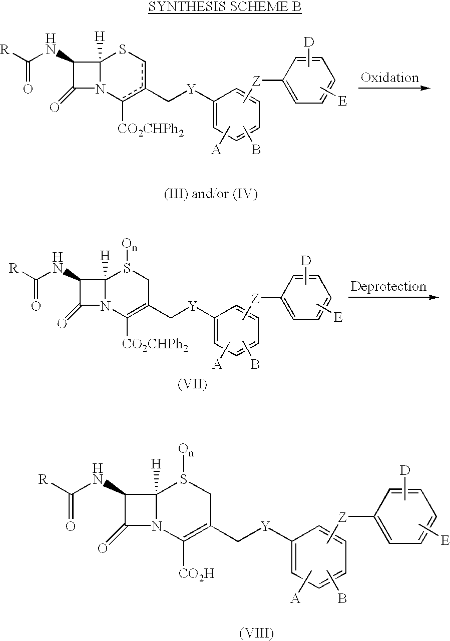

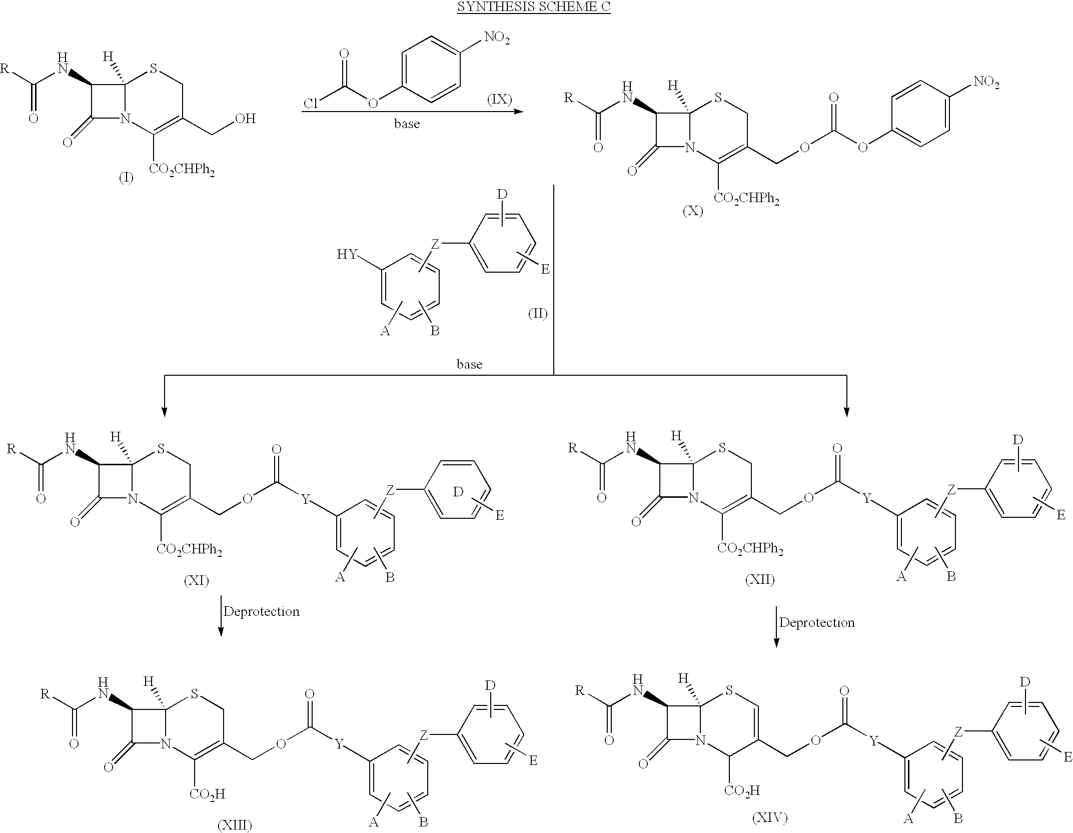

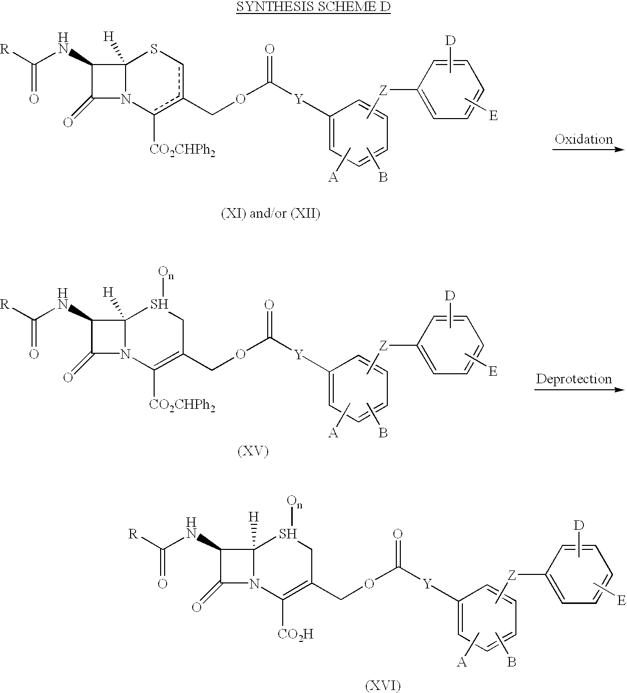

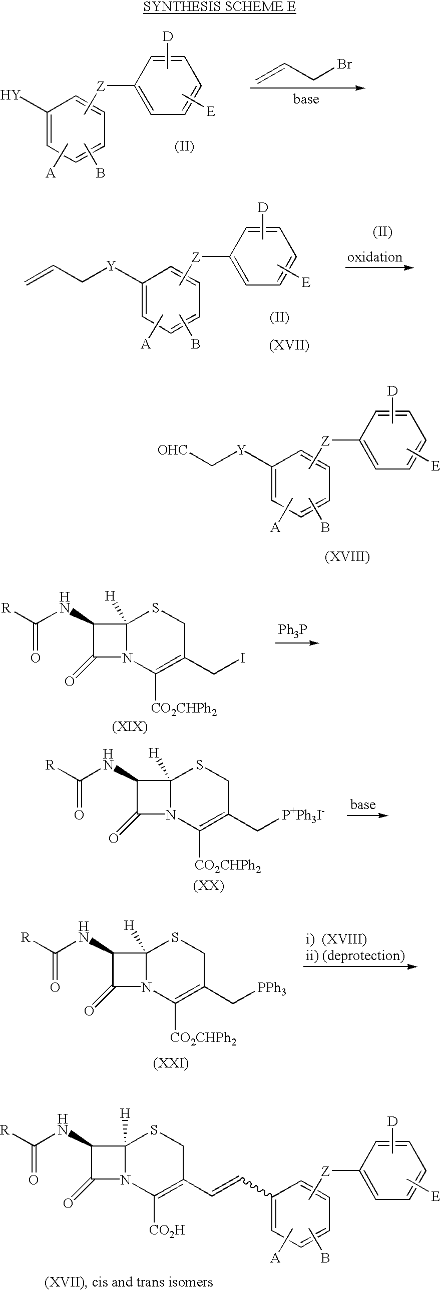

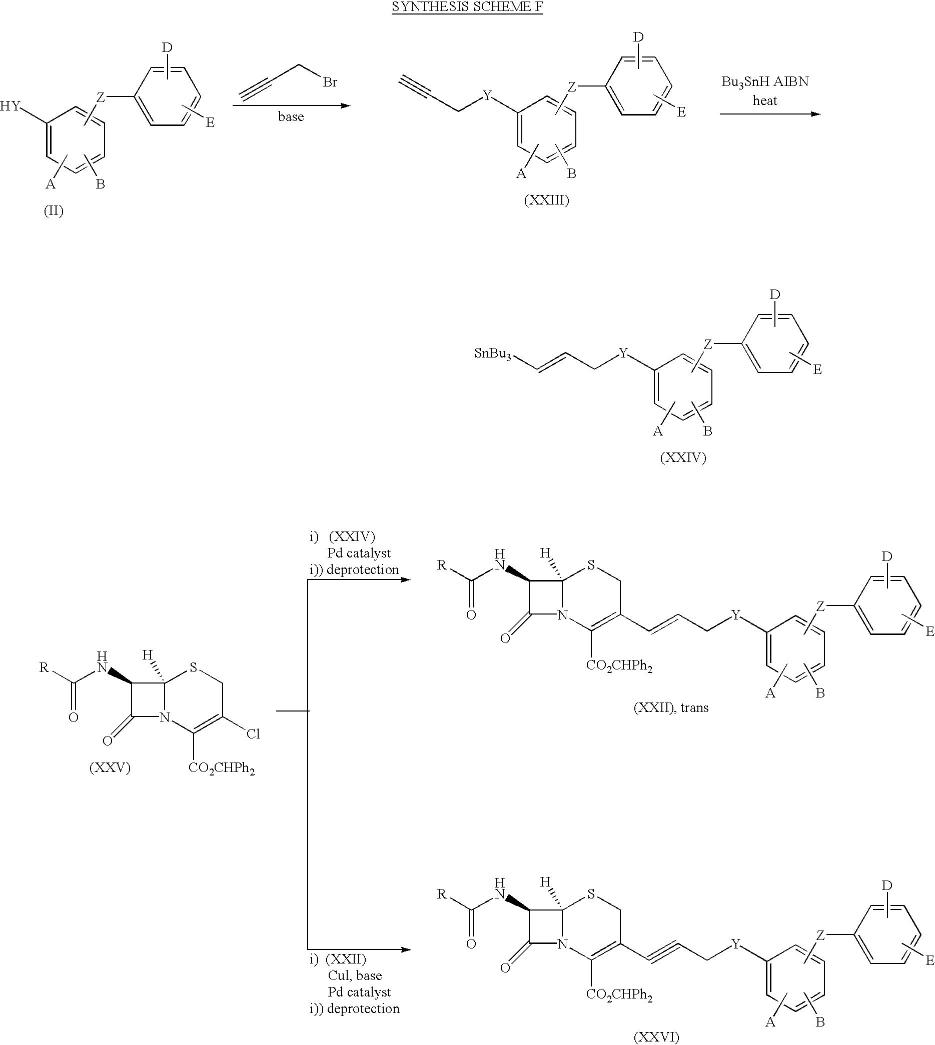

Specific Synthesis Schemes

The following are provided as specific examples chemical synthesis for the compounds of the invention.

### Example 1

### 3-(2-(2,4-Dichlorophenoxy)-5-chlorophenoxy)methyl-7-(2-thienylacetamido)-3-cephem-4-carboxylate (11)

(a) Diphenylmethyl 3-hydroxymethyl-7-(2-thienylacetamido)-3-cephem-4-carboxylate (5)

To a cooled (−5° C.) suspension of 7-aminocephalosporanic acid (1; 5 g, 18.4 mmoles) in MeOH (9.5 mL) is added 1 N NaOH (18.4 mL) over 30 minutes. Additional 1 N NaOH (18.4 mL) is then added over 7 minutes at 2°-5° C. Acetone (12.5 mL) is added to the cold solution followed by solid NaHCO₃ (4.63 g). 2-Thiopheneacetyl chloride (2.5 mL) is then added over 30 minutes at 0°-5° C. while maintaining the pH at 7 by simultaneous addition of NaHCO₃. The reaction is stirred at −5° C. for 2.5 hours. The mixture is washed with ethyl acetate (25 mL) and the layers were separated. The aqueous phase is layered with ethyl acetate (40 mL) and the resulting mixture is acidified at 0C. with concentrated HCl to pH 2-3. The layers are separated and the aqueous phase is extracted with ethyl acetate (40 mL). The ethyl acetate layer is filtered and the volatile is evaporated in vacuo to yield 5.1 g of crude 3-hydroxymethyl-7-(2-thienylacetamido)-3-cephem-4-carboxylic acid (3) as (77%) light brown solid.

A solution of diphenyldiazomethane (1.33 g, 6.6 mmole) in ethyl acetate (3 mL) is added to a cooled (5° C.) solution of (3) (2.13 g, 6.0 mmole) in 1:1 mixture of tetrahydrofuran/ethyl acetate (12 mL). The mixture is stirred until the purple color faded (ca. 4 hours) whereupon it is evaporated in vacuo. Tetrahydrofuran is added to the residue and the insoluble solid is filtered off (551 mg). The filtrate is evaporated in vacuo until crystals begin to form. Ethyl acetate is added and the mixture is stirred for 1.5 h at 0°-5° C. The resulting solid is filtered off (1.026 g). The solids are combined to provide the desired product (1.577 g, 57.6%) as a white solid.

(b) Diphenylmethyl 3-(2-(2,4-dichlorophenoxy)-5-chlorophenoxy)methyl-7-(2-thienylacetamido)-3-cephem-4-carboxylate (7) and diphenylmethyl 3-(2-(2,4-dichlorophenoxy)-5-chlorophenoxy)methyl-7-(2-thienylacetamido)-2-cephem-4-carboxylate (8)

To a stirring yellow suspension of (5) (520 mg, 1.0 mmol), triclosan (6) (293 mg, 1.0 mmol) and triphenylphosphine (265 mg, 1.0 mmol) in tetrahydrofuran is added diethyl azodicarboxylate (0.174 mL, 1.0 mmol) dropwise. The solution is stirred at 25° C. for 4 hours. The solvent is evaporated and the crude is purified by flask column chromatography on silica gel giving 595 mg of the 2 isomers of the desired product (75% yield, ca. 85% purity). The Δ³ isomer (7) can be isolated in pure form by repeated column chromatography. A mixture of the Δ² (8) and Δ³ (7) isomers is also obtained.

(c) 3-(2-(2,4-Dichlorophenoxy)-5-chlorophenoxy)methyl-7-(2-thienylacetamido)-3-cephem-4-carboxylic acid (9)

To a solution of the ester (7) (117 mg, 0.148 mmol) in methylene chloride (1 mL), anisole (0.02 mL) and trifluoroactetic acid (0.084 mL) arre added at 20° C. After 1 hour, the reaction is judged complete by TLC. The solvent is evaporated and the residue is stirred in cold diisopropyl ether for 10 minutes. The mixture is filtered and the solids are washed with cold diisopropyl ether and then dried in vacuo to give 41 mg of the desired product as a yellow solid.

### Example 2

### 3-(2-(2,4-Dichlorophenoxy)-5-chlorophenoxy)methyl-7-(2-thienylacetamido)-1-oxo-3-cephem-4-carboxylate (11)

(a) Diphenylmethyl 3-(2-(2,4-dichlorophenoxy)-5-chlorophenoxy)methyl-7-(2-thienylacetamido)-1-oxo-3-cephem-4-carboxylate (10)

A mixture of the Δ² and Δ³ esters (7) and (8) (140 mg, 0.178 mmol) dissolved in CH₂Cl₂ (1 mL) is cooled to 0° C. A solution of m-chloroperoxybenzoic acid (71% pure, 42.1 mg, 0.178 mmol) in CH₂Cl₂ (0.5 mL) is added and the mixture is stirred at 0° C. until the reaction is judged complete by TLC (1 hour). The desired product is isolated by flask column chromatography on silica gel to give 123 mg (86%) of (10) as a faint yellow solid.

(b) 3-(2-(2,4-Dichlorophenoxy)-5-chlorophenoxy)methyl-7-(2-thienylacetamido)-1-oxo-3-cephem-4-carboxylate (11)

The ester (10) is deprotected in a similar manner as described for compound (9) to give the desired product (11) as an off white solid.

### Example 3

### 3-((2-(2,4-Dichlorophenoxy)-5-chlorophenoxy)carbonyloxy)methyl-7-(2-thienylacetamido)-2-cephem-4-carboxylic acid (15)

(a) Diphenylmethyl 3-(4-nitrophenoxycarbonyloxy)methyl-7-(2-thienylacetamido)-3-cephem-4-carboxylate (12)

To a 0°C. suspension of compound (5) (520 mg, 1.0 mmol) in THF (1.5 mL) is added 4-(dimethylamino)pyridine (1 mg) and 4-nitrophenyl chloroformate (II) (316 mg. 1.5 mmol). 2,6-Lutidine (175 μL, 161 mg) is then added dropwise over ca. 3 minutes. The suspension is stirred while slowly being warmed up to 25° C. over two hours. After stirring overnight at 25° C., a thick paste is formed and stirring is not possible. The crude product is diluted with CH₂Cl₂ (6 mL), stirred vigorously for 5 minutes and filtered. The filtrate is concentrated to ca. 3 ml and loaded onto a silica gel column packed in 1% EtOAc/CH₂Cl₂ The column is eluted with 1-5% EtOAc/CH₂C1₂ to give 333 mg (49%) of the desired product as a very light yellow gum.

(b) Diphenylmethyl 3-((2-(2,4-dichlorophenoxy)-5-chlorophenoxy)carbonyloxy)methyl-7-(2-thienylacetamido)-3-cephem-4-carboxylate (13) and diphenylmethyl 3-((2-(2,4-dichlorophenoxy)-5-chlorophenoxy)carbonyloxy)methyl-7-(2-thienylacetamido)-2-cephem-4-carboxylate (14)

To a solution of (12) (309 mg, 0.451 mmol) in dry pyridine (2 mL) is added triclosan (6) (170 mg, 0.59 mmol) and two drops of diisopropylethylamine. The reaction is stirred at 25° C. for 20 hours. More triclosan (50 mg) is added and the dark solution is stirred for an additional 6 h. The solvent and volatile components are evaporated in vacuo to give 624 mg dark brown oil. This crude product is purified by column chromate on a silica gel with 1-5% EtOAc/CH₂Cl₂. as eluate to give a mixture of Δ² (13) and Δ³ (14) isomers (107 mg, 28%yield), a fraction containing essentially pure Δ² isomer (13) (98 mg, 26% yield) and 30 mg of recovered starting material (10% recovery).

(c) 3-((2-(2,4-Dichlorophenoxy)-5-chlorophenoxy)carbonyloxy)methyl-7-(2-thienylacetamido)-2-cephem-4-carboxylic acid (15)

This compound is prepared in the same manner as described in Example 1, section (c) from diphenylmethyl 3-((2-(2,4-Dichlorophenoxy)-5-chlorophenoxy)carbonyloxy)-methyl-7-(2-thienylacetamido)-2-cephem-4-carboxylate (13). The product is obtained as light tan solid.

### Example 4

### 3-((2-(2,4-Dichlorophenoxy)-5-chlorophenoxy)carbonyloxy)methyl-7-(2-thienylacetamido)-1-oxo-3-cephem-4-carboxylic acid (17)

(a) Diphenylmethyl 3-((2-(2,4-dichlorophenoxy)-5-chlorophenoxy)carbonyloxy)methyl-7-(2-thienylacetamido)-1-oxo-3-cephem-4-carboxylate (16)

A mixture of compounds (13) and (14) (105 mg, 0.126 mmol) from Example 3, section (b) is dissolved in CH₂Cl₂ (1 mL) and cooled to 0° C. 3-Chloroperoxybenzoic acid (71% pure, 30 mg) is added and the solution is stirred at 0° C. for 1 hour. Additional 3-chloroperoxybenzoic acid (5 mg) is added and after a further 1 hour at 0° C., the reaction is judged complete by TLC analysis. The solution is loaded onto a silica gel column and eluted with 1-5% EtOAc/CH₂Cl₂ to give (16) (62 mg) as an off-white solid, melting point 139-142° C. (Dec).

(b) 3-((2-(2,4-Dichlorophenoxy)-5-chlorophenoxy)carbonyloxy)methyl-7-(2-thienylacetamido)-1-oxo-3-cephem-4-carboxylic acid (17)

The solid obtained above (62 mg, 0.073 mmol) is suspended in CH₂Cl₂ (1.5mL) and anisole (0.3mL) and cooled to 0° C. Trifluoroacetic acid (0.3 mL) is added dropwise and a solution is formed. The solution is stirred at 0° C. for 1 hour. The solvent and volatile components are evaporated, first on a rotary evaporator and eventually in vacuo to give a light brown syrupy liquid. The liquid is dissolved in EtOAc (5 mL) and saturated NaHCO₃ (7 mL) is added. The two-phase mixture is vigorously stirred for five minutes at which time a fine precipitate has formed. This precipitate is filtered and the solid is dried in vacuo to give the desired product (18mg) an off-white solid.

### Example 5

### 3-(1-(2-(2,4-Dichlorophenoxy)-5-chlorophenoxy)-3-propenyl)-7-(2-thienylacetamido)-3-cephem-4-carboxylic acid (24)

(a) 4-Nitrobenzyl 3-(acetoxymethyl)-7-(2-thienylacetamido)-3-cephem-4-carboxylate (19)

To a stirring slurry of cephalothin sodium salt (18) (418 mg, 1.0 mmol) in DMF (2 mL) is added 4-nitrobenzylbromide (218 mg, 1.0 mmol). The mixture is stirred at room temperature for 1 hour and then warmed to 50° C., wherein the solids dissolve completely. Stirring is continued for another hour. The solvent is evaporated, then the brown solid is dissolved in EtOAc, washed with water, dried over MgSO₄ and the volatiles evaporated to give the desired product which is a white solid (496 mg, 96% yield).

(b) 4-Nitrobenzyl 3-(iodomethyl)-7-(2-thienylacetamido)-3-cephem-4-carboxylate (20)

A solution of the compound obtained above (19) (320 mg, 0.6 mmol) in anhydrous CH₂Cl₂ (4 mL) is treated with trimethylsilyliodide (0.137 mL, 0.66 mmol) at 20° C. for 1 hour. The solution is then sequentially washed with ice-cold solution of 10% Na₂S₂O₃, 10% NaHCO₃, brine and dried over MgSO₄. Evaporation of solvent yields the desired product (20) (306 mg, 85%) as a faint yellow solid.

(c) 1-((2-(2,4-Dichlorophenoxy)-5-chlorophenoxy)-3-propenal (22)

To a solution of triclosan (1.0 g, 3.94 mmol) in DMF (1 mL) is added allyl bromide (0.433 mL, 4.95 mmol) and K₂CO₃ (585 mg, 4.19 mmol). The mixture is stirred at 70° C. until completion. The solvent is evaporated then the residue is dissolved in ethyl acetate, washed with water and brine, dried over MgSO₄ and evaporated to give 2-(2,4-dichlorophenoxy)-5-chlorophenyl allyl ether (1.11 g, 98% yield) as off-white crystals.

2-(2,4-Dichlorophenoxy)-5-chlorophenyl allyl ether (550 mg, 167 mmol) is dissolved in 1:1 dioxane/H₂O (10 mL) and a 2.5% solution of osmium tetroxide in tert-butanol (0.5 mL, 0.04 mmole) is added with stirring over 5 minutes, causing the reaction mixture to change from colorless to dark brown. The reaction is stirred at 25° C. while finely powdered sodium metaperiodate (714 mg, 3.34 mmol) is added in portions over a period of 30 minutes. After 16 hours of stirring at 25° C., a tan colored slurry with white precipitate is formed. The solid is filtered off and washed repeatedly with ether. The combined filtrate and washing is separated from the aqueous layer, dried over Na₂SO₄ and evaporated. The product is purified by column chromatography on silica gel using 20-30% EtOAc/hexanes as eluent to give the product (22) (451 mg, 82% yield) as a thick, slightly greenish liquid.

(d) 4-Nitrobenzyl 3-(1-(2-(2,4-Dichlorophenoxy)-5-chlorophenoxy)-3-propenyl)-7-(2-thienylacetamido)-3-cephem-4-carboxylate (23)

A yellow solution of the iodide (20) obtained above (780 mg, 1.3 mmol) in acetone (20 mL) is stirred in the dark at room temperature and treated over 45 minutes with a solution of triphenylphosphine (690 mg, 2.6 mmol) in ethyl acetate (2 mL). The solution is stirred until completion at 0° C. The solvent is evaporated then the residue is redissolved in ethyl acetate. The resulting suspension is filtered and the filtered solid is washed with ethyl acetate then dried in vacuo to give a yellow solid (838 mg, 75% yield).

To a solution of solid obtained above (500 mg, 0.58 mmol) in methylene chloride (2.5 mL) is added (1-(2-(2,4-dichlorophenoxy)-5-chlorophenoxy)-3-propenal (22, 239 mg, 0.722 mmol) and aqueous saturated NaHCO₃ (2.5 mL) successively. The resultant brown mixture is stirred for 16 hours at ambient temperature. The pH is then adjusted to 9 with 1N NaOH and stirring is continued for 3 hours. The organic phase is separated from the aqueous phase and is washed with aqueous 10% NaHCO₃ solution and then with brine. The organic phase is washed with brine, dried over MgSO₄ and concentrated under reduced pressure. The product (23) is isolated as a white solid (165 mg, 33% yield) by column chromatography on silica gel using 1-5% EttOAc/CH₂Cl₂ as eluent.

(e) 3-(1-(2-(2,4-Dichlorophenoxy)-5-chlorophenoxy)-3-propenyl)-7-(2-thienylacetamido)-3-cephem-4-carboxylic acid (24)

The title compound is obtained by deprotecting the 4-nitrobenzyl ester (23) using zinc powder in acetic acid. The pure product is obtained from the crude product by triturating with diisopropyl ether to give an off-white solid.

### Example 6

### 3-(2-(2,4-Dichlorophenoxy)-5-chlorophenoxy)methyl-7-(1-tetrazoleacetamido)-3-cephem-4-carboxylic acid (29)

(a) Diphenylmethyl 3-(2-(2,4-dichlorophenoxy)-5-chlorophenoxy)methyl-7-β-(o-hydroxy)benzylidenamino -3-cephem-4-carboxylate (26)

A solution of compound (25) (0.52 g, 1.0 mmol) and triclosan (0.36 g, 1.25 mmol) in anhydrous THF (10 ml) is cooled to −20° C. in a dry-ice bath under argon atmosphere. A solution of triphenylphosphine (0.32 g, 1.25 mmol) in anhydrous THF (5.0 ml) is then added via a syringe. After 10 minutes, a solution of diisopropylazodicarboxylate (DIAD) (0.25 g, 1.25 mmol) in anhydrous THF (5.0 ml) is slowly added using a syringe pump over 40 minutes. The reaction mixture is then poured into water (2×30 mL) and extracted with ethylacetate (2×25 mL). Combined ethylacetate extracts are washed with water, brine and dried over Na₂SO₄. Removal of volatiles followed by purification on silica gel column using 5% ethyl acetate in dichloromethane provides compound (26) as light yellow solid. Yield=0.48 g, 43%. Melting point: 83-84° C.

H¹ NMR (CDCl₃, 500 MHz): δ 8.64 (1H, s), 7.48-7.23 (1H, m), 7.05 (1H, dd, J=Hz), 6.99-6.88 (H, m), 6.81 (1H, d, J=Hz), 6.59(1H, d, J=8.75 Hz), 5.36 (1H, d, J =4.8 Hz), 5.08 (1H, d, J=1.85 Hz), 4.95 (1H, d, J=13.6 Hz), 4.73 (1H, d, J=13.6 Hz), 3.38 (1H, d, J=18.5 Hz), 3.25 (1H, d, J=18.5 Hz).

(b) Diphenylmethyl-3-(2-(2,4-dichlorophenoxy)-5-chlorophenoxy)methyl-7-amino-3-cephem-4-carboxylate (27)

A solution of compound (26) (0.30 gr, 0.38 mmol), in methanol (5 mL) and ethylacetate (2 mL) is cooled in an icebath and Girards-T reagent (0.10 gr, 1.6 mmol) is added. Ice-bath is removed after 15 minutes and the stirring is continued for another 1.0 hour. All the volatiles are removed under reduced pressure and the residue is dissolved in CH₂Cl₂ (10 ml). The CH₂Cl₂ layer is washed successively with water (2×25 ml), brine and dried over Na₂SO₄. Removal of volatiles under the reduced pressure provids the compound (27) as a faintly yellow solid which is used for the next reaction without further purification. Yield=0.23 g, 92%. Melting point: 72° C.

H¹ NMR (CDCl₃, 500 MHz): δ 7.43-7.25 (11H, m), 7.06 (1H, d, J=11.25 Hz), 6.95-6.93 (3H, m), 6.80 (1H, S), 6.58 (1H, d, J=8.45 Hz), 4.89-4.87 (2H, m), 4.77 (1H, d, J=5.0 Hz), 4.72 (1H, d, J=13.15 Hz), 3.30 (1H, d, J=18.6Hz), 3.18 (1H, d, J=18.6 Hz).

(c) Diphenylmethyl 3-(2-(2,4-dichlorophenoxy)-5-chlorophenoxy)methyl-7-(1-tetrazoleacetamido)-3-cephem-4-carboxylate (28)

A solution of compound (27) (0.25 g, 0.37 mmol) in anhydrous CH₂Cl₂ (10 ml) DCC (0.077 g, 0.37 mmol) followed by 1-tetrazoleacetic acid (0.048 gr, 0.37 mmol) is added at room temperature. After 2.0 hours the reaction is filtered and purified on a short silica gel column using 10% EtOAc in CH₂Cl₂ as eluant to get compound (28) (0.26 gr, 89.3%) as white solid.

H¹ NMR (CDCl₃, 500 MHz): δ 8.78 (1H, s), 7.44 (1H, s), 7.40-7.26 (10H, m), 7.05-6.92 (4H, m), 6.81 (1H, S), 6.59 (1H, d, J=7.9 Hz), 5.81-5.79 (1H, m), 5.10 (2H, s), 4.92 (1H, d, J=7.9 Hz), 4.88 (1H, d, J=18.2Hz), 4.78 (1H, d, J=18.2 Hz), 3.35 (1H, d, J=17.3Hz), 3.14 (1H, d, J=17.3 Hz).

(d) 3-(2-(2,4-Dichlorophenoxy)-5-chlorophenoxy)methyl-7-(1-tetrazoleacetamido)-3-cephem-4-carboxylic acid (29)

Compound (28) (0.15 g, 0.19 mmol) is dissolved in anhydrous anisole (0.83 g, 7.68 mmol) and cooled to 0° C. in an ice bath. Trifluoroacetic acid (2.6 g, 23.12 mmol) is added and stirred for 1.0 hour under argon. All the volatile components are evaporated first on a rotary evaporator and eventually in vacuo to give a light brown solid. Ether is added and stirred for 1.0 hour at room temperature. The solid is filtered and dried in vacuo to give compound (29) (84 mg, 71%) as white solid. Melting point: 112° C.

H¹ NMR (DMSO-D₆, 500 MHz): δ 9.41 (1H, d, J=6.8 Hz), 9.36 (1H, s), 7.74 (1H, s), 7.71 (1H, s), 7.31-7.29 (1H, d, J=2.1 Hz), 7.10 (1H, d, J=8.9 Hz), 7.01-7.00 (1H, m), 6.74 (1H, d, J=7.3 Hz), 5.51-5.49 (1H, m), 5.34 (2H, d, J=8.2 Hz), 5.18 (1H, d, J=14.2 Hz), 4.88 (1H, d, J=7.3 Hz), 4.79 (1H, J=14.2 Hz), 3.12 (1H, d, J=17.3 Hz), 2.83 (1H, d, J=17.3 Hz).

### Example 7

### 3-(2-(2,4-Dichlorophenoxy)-5-chlorophenoxy)methyl-7-[2-(3H-imidazol-4-yl)]-acetamido-3-cephem-4-carboxylic acid (31)

(a) Diphenylmethyl 3-(2-(2,4-dichlorophenoxy)-5-chlorophenoxy)methyl-7-[2-(3-tert-butoxycarbonyl-3H-imidazol-4-yl)]-acetamido-3-cephem-4-carboxylate (30)

A solution of compound (27) (0.16 g, 0.24 mmol) and 4-carboxymethyl-imidazole-1-carboxylic acid tert-butyl ester (0.06 g, 0.24 mmol) is dissolved in anhydrous CH₂Cl₂ (10 mL) and DCC (0.05 g, 0.247 mmol) is added at room temperature. Reaction is complete after after stirring for 4 hours at room temperature. The precipitate is filtered off and the product purified on a short silicagel column using 20% EtOAc in CH₂Cl₂ to give compound (30) (0.17g, 82%) as a white solid.

H¹ NMR (CDCl₃, 500 MHz): δ 8.08 (1H, s), 7.97 (1H, d, J=7.33), 7.44-7.43 (2H, m), 7.36-7.25 (10H, m), 7.06-7.04 (1H, m), 6.96 (1H, s), 6.90 (1H, s), 6.83 (1H, s), 6.59 (1H, s), 5.91 (1H, m), 4.95-4.91 (2H, m), 4.76 (1H, d, J=13.8 Hz), 3.58 (2H, s), 3.30 (1H, d, J=18.5 Hz), 3.20 (1H, d, J=18.5 Hz), 1.60 (9H, s).

(b) 3-(2-(2,4-Dichlorophenoxy)-5-chlorophenoxy)methyl-7-[2-(3H-imidazol-4-yl)]-acetamido-3-cephem-4-carboxylic acid (31)

This compound is prepared by using general benzhydryl group deprotecting method as described for the synthesis of compound (29).

H¹ NMR (DMSO-D₆, 500 MHz): 67 9.20 (1H, s), 8.88 (1H, s), 7.77 (1H, s), 7.43 (1H, s), 7.35 (1H, d, J=2.1Hz), 7.32-7.30 (1H, m), 7.14 (1H, s), 7.14-7.08 (1H, m), 6.81 (1H, d, J=7.3 Hz), 5.74 (1H, m), 5.07 (1H, d, J=8.2 Hz), 4.98 (1H, d, J=14.8 Hz), 4.80 (1H, d, J=14.8 Hz), 3.78 (2H, s), 3.34 (1H, d, J=18.5 Hz), 3.12 (1H, d, J=18.5 Hz).

### Example 8

### Compound(32)

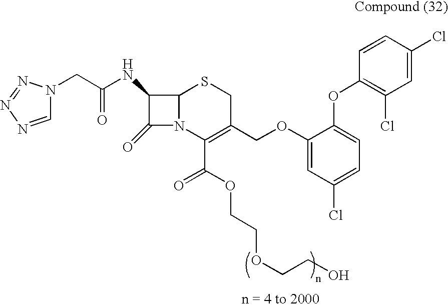

To a solution of compound (29) (0.1 g, 0163 mmol) and hydroxy benzothiozole (22 mg, 0.163 mmol) in anhydrous THF (5.0 ml), DCC (33 mg, 0.163 mmol) is added. Tetraethylene glycol (1.0 g) is added and stirred at room temperature for 2 hours. The reaction mixture is then filtered and passed through a short bed of silicagel to get compound (32) (0.11 g, 87%) as a gum.

### Example 9

### {4-Chloro-2-[2-(3,4-dichloro-phenyl)-acetyl]-phenoxy}-acetaldehyde (33)

Compound (33) is prepared from 1-(5-chloro-2-hydroxy-phenyl)-2-(3,4-dichloro-phenyl)-ethanone by a procedure used for the synthesis of compound (22). Compound (32) is isolated as a white solid (77% yield). IR (neat): 1655 cm⁻¹, 1670 cm⁻¹, 1590 cm⁻¹.

### Example 10

### 3-{3-[4-Chloro-2-(3,4-dichloro-phenylcarbamoyl)-phenoxy]-propenyl}-7-(2-thiophene-acetmido)-3-cephem-4-carboxylic acid (35)

Compound (35) was prepared using the procedure for the synthesis of compound (23).

H¹ NMR (DMSO-D₆, 500 MHz): 67 10.48 (1H, s), 9.14 (1H, d, J=5.8 Hz), 8.10 (1H, s), 7.60-7.54 (3H, m), 7.37 (1H, d, J=2.6 Hz), 7.13 (1H, d, J=5.1 Hz), 6.96-6.93 (2H, m), 6.54 (1H, s) 5.79-5.76 (1H, m), 5.69 (1H, m), 5.15 (1H, d, J=3.2 Hz), 4.83-4.77 (1H, m), 4.68-4.65 (1H, m), 3.77 (2H, s), 3.74 (1H, d, J=18.5 Hz), 3.55 (1H, d, J=18.5 Hz).

### Example 11

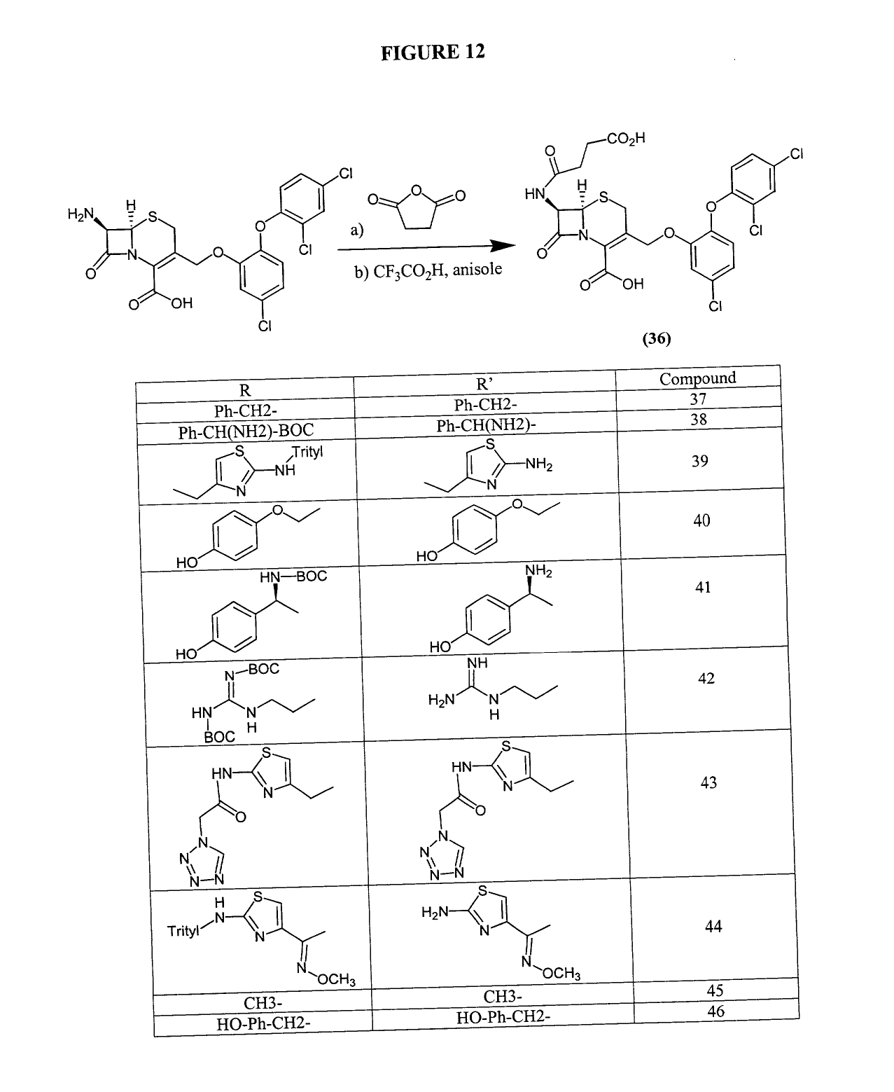

FIG. 12, duplicated here, shows the specific synthesis scheme for compound (36).

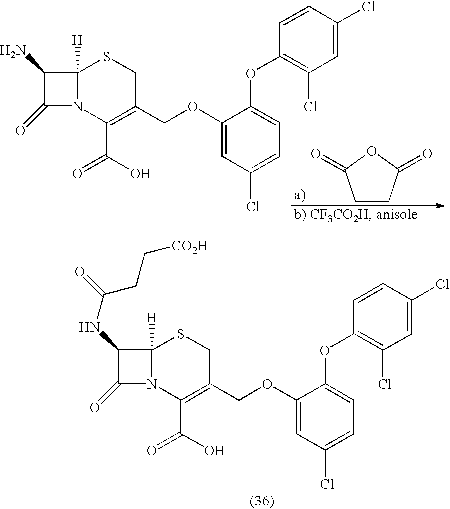

### Example 12

The following is the general synthetic scheme for compounds 37 through 47, with an accompanying table to indicate which carboxylic acids are used to form each particular compound. Below the compounds 37 through 43 are listed individually with their NMR parameters.

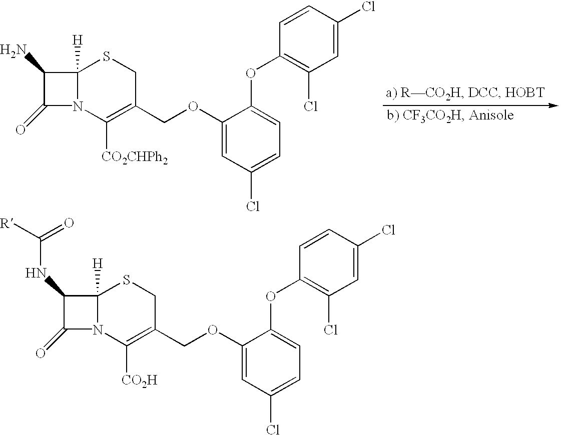

| R | R′ | Compound |
|---|---|---|
| Ph—CH2— | Ph—CH2— | 37 |
| Ph—CH(NH2)-BOC | Ph—CH(NH2)— | 38 |
| 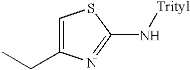 | 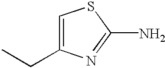 | 39 |
| 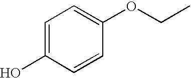 | 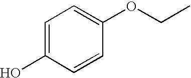 | 40 |
| 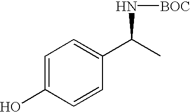 | 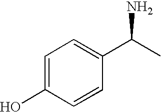 | 41 |
| 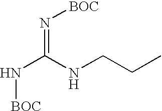 | 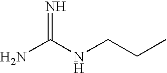 | 42 |
| 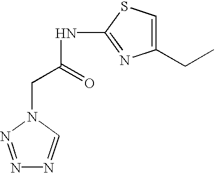 | 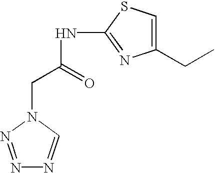 | 43 |
| 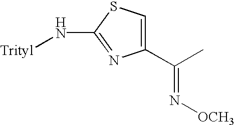 | 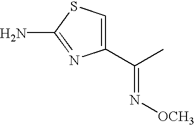 | 44 |
| CH3— | CH3— | 45 |
| HO—Ph—CH2— | HO—Ph—CH2— | 46 |
| 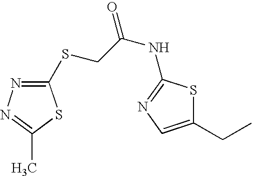 | 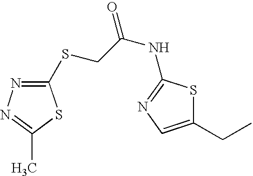 | 47 |

(a) 3-(2-(2,4-Dichlorophenoxy)-5-chlorophenoxy)methyl-7-(1-phenyl-2-aminoacetamido)-3-cephem-4-carboxylic acid (37)

The following description is the synthesis for compounds (37) through (47). To a solution of compound (27) (1.0 mmol) in anhydrous THF (20 mL) hydroxybenzothiazole (1.0 mmol), DCC (1.0 mmol) are added at under argon atmosphere. After 30 minutes the carboxylic acid part (R—CO₂H) (See above chart for correct carboxylic acid) (1.0 mmol) is added and stirred until the completion of the reaction. The formed precipitate is filtered and the crude product is passed through a short bed of silica gel to get the appropriate amide product in 85 to 95% yields.

H¹ NMR (DMSO-D₆, 500 MHz): 67 9.50 (1H, d, J=2.7 Hz), 7.68 (1H, s), 7.52-7.43 (8H, m), 7.25 (1H, d, J=2.1 Hz), 7.12 (1H, d, J=3.3 Hz), 7.08 (1H, d, J=3.1 Hz), 6.74 (1H, d, J=3.55 Hz), 5.70 (1H, brs), 5.02-4.98 (3H, m), 4.71 (1H, d, J=14.8 Hz), 3.72 (1H, d, J=2.5 Hz), 3.15 (1H, d, J=7.19 Hz), 2.85 (1H, d, J=7.19 Hz).

(b) 3-(2-(2,4-Dichlorophenoxy)-5-chlorophenoxy)methyl-7-(1-phenyl-2-aminoacetamido)-3-cephem-4-carboxylic acid (38)

H¹ NMR (DMSO-D₆, 500 MHz): 67 9.50 (1H, d, J=2.7 Hz), 7.68 (1H, s), 7.52-7.43 (8H, m), 7.25 (1H, d, J=2.1 Hz), 7.12 (1H, d, J=3.3 Hz), 7.08 (1H, d, J=3.1 Hz), 6.74 (1H, d, J=3.55 Hz), 5.70 (1H, brs), 5.02-4.98 (3H, m), 4.71 (1H, d, J=14.8 Hz), 3.72 (1H, d, J=2.5 Hz), 3.15 (1H, d, J=7.19 Hz), 2.85 (1H, d, J=7.19 Hz).

(c) 3-(2-(2,4-Dichlorophenoxy)-5-chlorophenoxy)methyl-7-[4-(2-aminothiazole)-yl-2-acetamido]-3-cephem-4-carboxylic acid (39)

H¹ NMR (DMSO-D₆, 500 MHz): 67 8.96 (1H, s), 7.71 (1H, s), 7.35 (1H, s), 7.30 (1H, dd, J=1.9, 8.8 Hz), 7.15 (1H, d, J=8.6 Hz), 7.08 (1H, d, J=2.0 Hz), 6.74-6.72 (1H, m), 6.66 (1H, m) 5.71-5.70 (1H, m), 5.05 (1H, brs), 4.97 (1H, d, J=4.9 Hz), 4.90 (1H, s), 4.80 (1H, d, J=12.06 Hz), 4.48 (2H, q), 3.09 (1H, d, J=14.4 Hz)

(d) 3-(2-(2,4-Dichlorophenoxy)-5-chlorophenoxy)methyl-7-12-(4-hydroxyphenoxy)acetamido-3-cephem-4-carboxylic acid (40)

H¹ NMR (DMSO-D₆, 500 MHz): 67 9.05 (1H, s), 7.72 (1H, s), 7.35 (1H, s), 7.30 (1H, d, J=3.5 Hz), 7.14 (1H, s), 7.10 (1H, d, J=3.5 Hz), 6.80 (1H, d, J=3.5 Hz), 6.52 (1H, s) 5.71 (1H, dd, J=2.9 Hz, 3.2 Hz), 5.05 (1H, d, J=1.9 Hz), 4.97 (1H, d, J=4.9 Hz), 4.80 (1H, d, J=4.9 Hz), 3.89 (2H, s), 3.31 (1H, d, J=7.3 Hz), 3.10 (1H, d, J=17.3 Hz)

(e) 3-(2-(2,4-Dichlorophenoxy)-5-chlorophenoxy)methyl-7-[2-amino-2-(4-hydroxy-phenyl)acetamido)-3-cephem-4-carboxylic acid (41)

H¹ NMR (DMSO-D₆, 500 MHz): δ 9.47 (1H, s), 7.68 (1H, d, J=2.1 Hz), 7.62 (1H, brs), 7.28 (1H, dd, J=1.9, 8.82 Hz,), 7.19 (1H, s), 7.17 (1H, s), 7.12 (1H, d, J=8.2 Hz), 7.02 (1H, dd, J=2.0, 8.31 Hz), 6.74-6.70 (3H, m), 4.92 (1H, d, J=11.3 Hz), 4.69 (1H, d, J=11.3 Hz), 4.52 (1H, brs), 4.38 (1H, brs), 3.14 (1H, d, J=17.0 Hz), 2.72 (1H, d, J=17.0 Hz)

(f) 3-(2-(2,4-Dichlorophenoxy)-5-chlorophenoxy)methyl-7-(3-guanidinopropyl)acetamido-3-cephem-4-carboxylic acid (42)

H¹ NMR (DMSO-D₆, 500 MHz): δ 8.84 (1H, d, J=8.2 Hz), 8.21(1H, d, J=7.71 (1H, d, J=2.0 Hz), 7.34 (1H, d, J=2.0 Hz), 7.30 (1H, dd, J=2.0, 8.2 Hz), 7.14 (1H, s), 7.10-7.07 (1H, m), 6.80 (1H, d, J=8.85 Hz), 5.68 (1H, dd, J=4.82, 8.09 Hz), 5.04 (1H, d, J=4.80 Hz), 4.96 (1H, d, J=12.27 Hz), 4.80 (1H, d, J=12.27 Hz), 3.89 (2H, s), 3.31 (1H, d, J=7.3 Hz), 3.10 (1H, d, J=17.3 Hz)

(g) 3-[5-Chloro-2-(2,4-dichlorophenoxy)-phenoxymethyl]-7-{2-[2-(2-tetrazol-1-yl-acetamido)-thiazol-5-yl]-acetamido-3-cephem-4-carboxylic acid (43)

H¹ NMR (DMSO-D₆, 500 MHz): 67 9.42 (1H, s), 7.71 (1H, d, J=2.0 Hz), 7.34 (1H, d, J=2.0 Hz), 7.30 (1H, dd, J=2.0, 8.2 Hz), 7.14 (1H, s), 7.10-7.07 (1H, m), 6.80 (1 H, d, J=8.85 Hz), 5.68 (1H, dd, J=4.82, 8.09 Hz), 5.04 (1H, d, J =4.80 Hz), 4.96 (1H, d, J=12.27 Hz), 4.80 (1H, d, J=12.27 Hz), 3.89 (2H, s), 3.31 (1H, d, J=7.3 Hz), 3.10 (1H, d, J=17.3 Hz)

### Example 13

Enzyme Assay

A typical TEM-1 β-lactamase assay is conducted at 37° C. in 1.4 ml of 100 mM potassium phosphate pH 7.2, 1 mM; ethylenediamine tetraacetic acid (EDTA) and 25 nM TEM-1 β-lactamase. A test compound is assayed at several concentrations, for example at 5 and 20 μM. At timed intervals oft=0, 15, 30, 60 and 120 minutes, 200 μl of the assay mixture is withdrawn and added to 8 μl of 10% trifluoracetic acid (TFA) in an high pressure liquid chromatography.(HPLC) microvial. Substrates and products are separated and quantitated via HPLC (described below). As a control for compound stability, the compound is assayed in the above solution in the absence of enzyme.

### Example 14

Kinetic Analysis

Kinetic parameters of test compounds (kcat and Km) are determined from a simultaneous progress curve analysis of substrate consumption and product formation (when both are observable) as fit to the equations:

[S]'=-kcat[E][S]/([S]+Km) and

[P]'=kcat[E](Sₒ-[P])/(Sₒ-[P]+Km).

[S]' is the differential of compound concentration with respect to time.

[P]' is the differential of product concentration with respect to time.

Sₒ is the concentration of compound at t =0.

[S] is the concentration of compound at time t. [P] is the concentration of product at time t.

[E] is the concentration of enzyme.

t=time in minutes.

The parameters are kcat (kinetic rate constant) and Km (the Michaelis constant) of TEM-1 β-lactamase for the compound being examined. Least squares fitting is done via the software Scientist Version 2.0 Micromath Scientific Software, Salt Lake City, Utah.

### Example 15

HPLC analysis

75 μl of a sample is injected onto an Agilent zorbax 3.5 μm 4.6×75 mm column under the following mobile phase conditions (solvents A and B): 1 ml/min 0% B for 1 min, gradient from 0% to 55% B in 4 min, isocratic at 55% B for 5 min, gradient from 55% to 100% B in 5 min, isocratic at 100% B for 3 min, gradient from 100% B to 0% B in 0.5 min, isocratic at 0% B for 1.5 minutes. Solvent A=H₂O with 0.1% TFA, Solvent B=acetonitrile with 0.1% TFA. Detection and quantitation is via peak area at 280 nm. Concentrations are determined via standards.

### Example 16

Serum Stability Assay

200 μM of test compound is incubated at 37° C. in 1.5 ml of human male AB serum. At timed intervals, 200 μl are withdrawn and quenched with 800 μl acetonitrile. Samples are mixed and centrifuged to remove pelleted protein. Supernatant is evaporated via centrifugation under vacuum. The remaining material is resuspended with 50 μl DMSO and taken to 500 μl with 100 mM potassium phosphate pH 7.2, 1 mM EDTA and analyzed via HPLC as described above.

### Example 17

Susceptibility Testing

Methods

The NCCLS (National Committee for Clinical Laboratory Standards) method to determine MIC's of antimicrobial compounds was modified for high-throughput screening. All stocks of tested compounds were prepared in either water or in dimethylsulfoxide (DMSO), depending on the solubility. At the highest tested concentration, DMSO content did not exceed 0.5%. Briefly 20 2-fold serial dilutions of testing compounds from the highest concentration were made in a 384-well microtiter plate. Each well was inoculated with testing bacteria in broth to a final concentration of approximately 1-1.5×10⁶ cells/ml. Bacterial growth was determined by the increase of optical density at 600 nm using a microplate reader (Tecan SpectraFluor Plus). The MIC was defined as the lowest concentration at which bacteria growth was inhibited after 16-18 hours of incubation at the appropriate temperature required for the bacteria growth.

Results

| | | MIC μg/ml | | | | | | | |
|---|---|---|---|---|---|---|---|---|---|
| ATCC # | ORGANISM | 11 | 37 | 17 | 48 | 24 | 38 | 39 | 49 |
| | E. coli (N) | | 4 | 0.016 | 1 | 0.26 | 0.031 | 1 | 4.12 |
| | E. coli/Tem-1 | | 0.12 | 0.032 | 0.016 | 0.016 | 0.015 | 0.063 | 1.03 |
| 700260 | S. aureus | 0.0078 | 0.0039 | 6.3E−05 | 0.001 | 1.0E−05 | 1.5E−05 | 0.002 | 0.032 |
| 700699 | S. aureus | 1 | | 2.5E−4 | 0.13 | 0.032 | 1.5E−05 | 4.0 | 4.12 |
| 11632 | S. aureus | | | | | 0.065 | 0.062 | | |
| 33591 | S. aureus | | | | | | | | |
| 33594 | S. aureus | | | | | 0.065 | 0.062 | | |
| 700802 | E. faecalis | | | | | 8.28 | 15.9 | | |
| 49757 | E. faecalis | | | | 16.5 | 4.14 | 15.9 | 16 | >16.5 |
| 23355 | E. cloacae | | | | | 2.07 | 1.98 | | |
| 35028 | E. aerogenes | | | | 4.1 | 4.14 | 1.98 | >16 | >16.5 |
| 51503 | K. pneumoniae | | | | 2.06 | 2.07 | 1.98 | 16 | >16.5 |
| 700721 | K. pneumoniae | 4 | 8 | 1.03 | | 2.07 | | | |
| 29872 | P. aeruginosa | >64 | | | | >16.6 | >15.9 | | |
| 21726 | P. aeruginosa | >64 | >16 | >16.5 | >16.5 | >16.6 | >15.9 | >16 | >16.5 |

| | | MIC μg/ml | | | | | | | | |
|---|---|---|---|---|---|---|---|---|---|---|
| ATCC# | ORGANISM | 15 | 50 | 41 | 51 | 29 | 46 | 35 | 52 | 44 |
| | E. coli (N) | 0.008 | 0.0041 | 0.25 | 4 | 2 | 16 | | | |
| | E. coli/Tem-1 | 0.0031 | 0.0041 | 0.25 | 0.063 | 0.063 | 1 | | | |
| 700260 | S. aureus | 0.061 | 0.065 | 0.063 | 0.063 | 0.063 | 0.063 | 0.25 | 8 | 0.063 |
| 700699 | S. aureus | 0.061 | 0.065 | 0.063 | 0.125 | 0.125 | 0.063 | >32 | >32 | >32 |
| 11632 | S. aureus | 0.61 | 0.065 | 0.063 | 0.125 | 0.063 | 0.063 | 0.063 | 1 | 0.5 |
| 33591 | S. aureus | | | | | | | | | |
| 33594 | S. aureus | 0.061 | 0.065 | 0.063 | 0.063 | 0.063 | 0.063 | 0.063 | 4 | 0.063 |
| 700802 | E. faecalis | 15.7 | 16.9 | 32 | >32 | 16 | 32 | >32 | >32 | 32 |
| 49757 | E. faecalis | >15.7 | >16.9 | 32 | >32 | 8 | >32 | 8 | >32 | 32 |
| 23355 | E. cloacae | 0.25 | 0.26 | 1 | 4 | 1 | 2 | >32 | >32 | 4 |
| 35028 | E. aerogenes | 0.98 | 0.26 | 4 | 32 | 32 | 4 | >32 | >32 | >32 |
| 51503 | K. pneumoniae | 1.96 | 1.06 | 8 | >32 | 8 | 32 | >32 | >32 | 8 |
| 700721 | K. pneumoniae | 1.96 | 0.53 | 8 | 4 | 1 | 8 | >32 | >32 | 8 |
| 29872 | P. aeruginosa | >15.7 | >16.9 | >32 | >32 | >32 | >32 | >32 | >32 | >32 |
| 21726 | P. aeruginosa | >15.7 | >16.9 | >32 | >32 | >32 | >32 | >32 | >32 | >32 |

| | | MIC | | | | | | | | |
|---|---|---|---|---|---|---|---|---|---|---|
| ATCC# | ORGANISM | 45 | 53 | 47 | 43 | 40 | 54 | 31 | 42 | 36 |
| | E. coli (N) | | | 2 | 2 | 0.5 | | 0.25 | 1 | |
| | E. coli/Tem-1 | | | 0.25 | 0.25 | 0.063 | | 0.063 | 0.125 | |
| 700260 | S. aureus | 0.063 | 0.5 | 8 | 8 | 4 | 0.063 | 32 | 16 | >32 |
| 700699 | S. aureus | 16 | >32 | >32 | 32 | 32 | 16 | 32 | >32 | >32 |
| 11632 | S. aureus | >32 | 32 | >32 | 32 | 16 | 16 | 16 | >32 | >32 |
| 33591 | S. aureus | | | 0.125 | 0.125 | 0.125 | 0.063 | 16 | 8 | 2 |
| 33594 | S. aureus | 0.063 | 0.063 | 0.063 | 0.063 | 0.063 | 8 | 0.063 | 0.063 | >32 |
| 700802 | E. faecalis | 32 | 32 | >32 | 32 | 16 | 4 | >32 | >32 | >32 |
| 49757 | E. faecalis | 16 | >32 | >32 | 32 | 16 | >32 | 32 | >32 | >32 |
| 23355 | E. cloacae | >32 | >32 | >32 | 32 | 16 | 16 | 16 | >32 | >32 |
| 35028 | E. aerogenes | >32 | >32 | >32 | 32 | 32 | 32 | 16 | >32 | >32 |
| 51503 | K. pneumoniae | 8 | >32 | >32 | 32 | 16 | 8 | 32 | 16 | >32 |
| 700721 | K. pneumoniae | 2 | >32 | 8 | 4 | 2 | 16 | 0.5 | 4 | >32 |
| 29872 | P. aeruginosa | >32 | >32 | >32 | >32 | >32 | >32 | >32 | >32 | >32 |
| 21726 | P. aeruginosa | >32 | >32 | >32 | >32 | >32 | >32 | >32 | >32 | >32 |

### Example 18

Inhibition of Bacterial Growth by Compounds (9) and (11) as Compared with Triclosan and Ampicillin

Compounds of the invention were tested for their ability to inhibit the growth of β-lactam sensitive and resistant bacteria by contacting samples of such cells with various dilutions of the compositions, incubating the cells at 37° C. in the presence of these compositions and then measuring the number of viable cells present in each sample.

Two compounds of the invention were tested, Compound (9) and Compound (1l). Incubation of bacterial cells was performed for two time periods, 10 hours and 20 hours. Four E. coli strains were used for the test. These strains were all derived from the strain NovaBlue (NovaGen Inc., Madison, Wis.). E. coli N is a normal, β-lactam sensitive strain. E coli R(Tem1) is a β-lactam resistant strain with a β-lactamase gene on a plasmid. E. coli C(Tem4O-15) is a β-lactam resistant strain with a β-lactam inhibitor resistant β-lactamase gene, and E. coli C(Tem3 1-27) is β-lactam resistant strain with an alternative β-lactam inhibitor resistant β-lactamase gene.

The various E. coli strains were each treated with dilutions of the compounds of the invention. For purposes of comparison, antibiotic sensitive and resistant E. coli cells were also treated with the β-lactam antibiotic ampicillin and the antibacterial agent triclosan (the substance, which is the toxophore, released from the compositions of the invention upon catalysis by β-lactamase enzyme). The number of viable cells at each time point was determined by measuring the OD₆₀₀ of each cell sample.

After measuring the number of viable cells present, the IC₅₀ for each sample was determined. Results are presented in Table 2, below. Both compounds were found to be significantly more effective than ampicillin at inhibiting the growth of the bacterial cells. In addition, both compounds were effective at inhibiting the growth of both antibiotic sensitive and resistant bacterial cells.

TABLE 2

| Cell | Compound | IC50 10 hours | | | IC50 20 hours | | |
|---|---|---|---|---|---|---|---|
| N | Compound (9) | 189.1 | nM | | 42.4 | nM | |
| R (Tem1) | Compound (9) | 0.096 | nM | N/R = 1969x | 4.13 | nM | N/R = 10x |
| C (Tem40-15) | Compound (9) | 1.62 | nM | C/R = 117x | 18.3 | nM | C/R = 2.3x |
| C (Tem31-27) | Compound (9) | 2.09 | nM | C/R = 90x | 21.0 | nM | C/R = 2x |
| N | Triclosan | 3.37 | nM | | 1.25 | nM | |
| R (Tem1) | Triclosan | 0.24 | nM | | 2.34 | nM | |
| N | Ampicillin | 9705 | nM | | | | |
| N | Compound 11 | 7480 | nM | | 4241 | nM | |
| R (Tem1) | Compound 11 | 5.39 | nM | N/R = 1388x | 35.3 | nM | N/R = 120x |
| C (Tem40-15) | Compound 11 | 369 | nM | N/C = 20x | 653 | nM | N/R = 6.5x |
| C (Tem31-27) | Compound 11 | 1111 | nM | N/C = 6.7x | 1477 | nM | N/R = 2.9x |
| N | Triclosan | 259 | nM | | 22.4 | nM | |
| R (Tem1) | Triclosan | 0.166 | nM | N/R = 1555x | 8.8 | nM | N/R = 2.5x |
| N | Ampicillin | 7880 | nM | | 7467 | nM | |

### Example 19

Compound (9): Synthesis and Bacterial Inhibition Properties

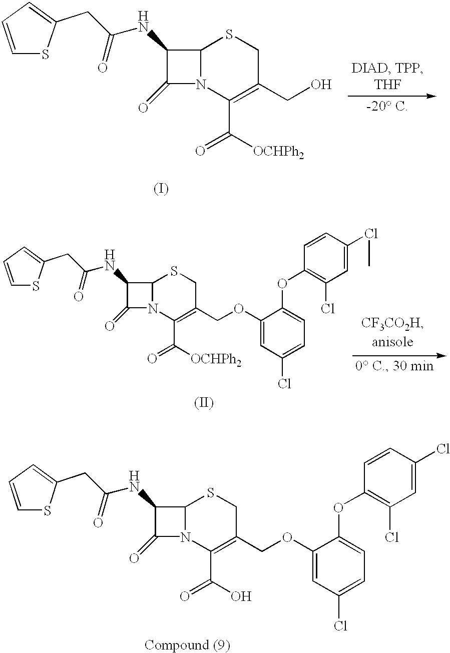

(a) Diphenylmethyl 7-(2-thienylacetylamino)-3-[5-chloro-2-(2,4-10 dichlorophenoxy)phenoxymethyl}-3-cephem-4-carboxylate (Compound (II) in synthesis scheme above).

A solution of diphenylmethyl 7-(2-thienylacetylamino)-3-hydroxymethyl-3-cephem-4-carboxylate (compound (I) in synthesis scheme above) (See U.S. Pat. No. 5,801,242) (0.52 g, 1.0 mmol) and triclosan (0.36 g, 1.25 mmol) in anhydrous THF (10 mL) was cooled to −20° C. in a dry-ice bath under an argon atmosphere. A solution of triphenylphosphine (0.32 g, 1.25 mmol) in anhydrous THF (5.0 mL) was then added via a syringe. After 10 minutes, a solution of diisopropyl diazodicarboxylate (DIAD; 0.25 g, 1.25 mmol) in THF (5.0 mL) was slowly added using a syringe pump over 40 minutes. The reaction mixture was then poured into water and extracted with ethyl acetate (2×25 ml). The combined ethyl acetate extracts were washed sequentially with water and brine and dried over Na₂SO₄. Removal of volatiles followed by purification on a silica gel column using 5% ethyl acetate in dichloromethane provided compound (II) as light yellow solid; yield =0.32 g, 32%.

(b) [7-(2-Thienylacetylamino)-3-[5-chloro-2-(2,4-dichlorophenoxy)phenoxymethyl]-3-cephem-4-carboxylic acid] (Compound (9))

Compound (II) (0.75 g, 0.94 mmol) was dissolved in anhydrous anisole (4.0 mL) and cooled in an ice bath under an argon atmosphere. Trifluoroacetic acid (9.0 mL) was then slowly introduced over 10 minutes. After 45 minutes, the volatiles were removed under reduced pressure with continuous stirring to give a light yellow residue. Ether (10 mL) was added and the mixture was stirred at 0° C. for one hour. The resultant light yellow precipitate was filtered, washed with cold ether (10 mL) and dried under vacuum to get Compound (9) as a light yellow solid, melting point 105° C. The yield was 0.37 g (64%).

¹H NMR DMSO-d6, 500 MHz): δ 3.10 (1H, d, J=16.5 Hz), 3.75 (2H, q_{AB}, J=15.17, 4.75 Hz), 4.80 (1H, d, J 12.0 Hz), 4.97 (1H, d, J=12.0 Hz), 5.03 (1H, d, J=4.0 Hz), 6.80 (1H, d, J=8.7 Hz), 6.92-6.95 (2H, m), 7.09-7.07 (1H, m), 7.16 (1H, d, J=8.7 Hz), 7.29-7.31 (1H, m), 7.31-7.37(1H, m), 7.72 (1H, d, J=2.8 Hz), 9.11(1H, d, J=8.3 Hz). IR (Neat): 1767, 1715, 1662, 1495 cm⁻¹. The purity of Compound (9) was 98.2% as determined by HPLC.

TEM-1 β-Lactamase Preparation

The N-terminal His-tagged TEM-1 construct TEM-1/pET28b(+) was generated by subcloning TEM-1 into Nco I and Hind III sites of pET28b(+) vector. TEM-1 was prepared by transforming TEM-1/pET28b(+) into the Escherichia coli BL21(DE3) (Novagen, Madison, Wis.) strain. Following induction with IPTG, TEM-1 was purified by affinity chromatography on a Ni²⁺His bind metal chelation resin (Novagen). The Ni²⁺His bind metal chelation column was washed with 20 mM Tris pH7.9, 5 mM imidazole, 0.5 M NaCl. TEM-1 was eluted with 20 mM Tris, pH 7.9, 100 mM imidazole and 0.5 M NaCl at room temperature. Purified TEM-1 was dialyzed against 100 mM Tris, pH 8.0. Aliquots were stored at −80° C.

Enzyme Assay of Compound (9)

All enzyme assays were conducted at 37° C. in an assay cocktail containing 100 mM potassium phosphate pH 7.0, 1 mM EDTA and 0.5 μg/ml (12.5 nM) of β-lactamase TEM-1. Initial assessment of purified β-lactamase TEM-1 was by use of the chromophoric substrate nitrocefin on a Tecan Spectrafluor Plus with a 495 cutoff filter on the excitation path. The molar absorptivity (15,900 M⁻¹ cm⁻¹) of the hydrolyzed product was determined from a standard curve of the total hydrolysis of nitrocefin. Enzyme catalysis of Compound (9) was determined by fixed time assays of varied concentrations of Compound (9) followed by the addition of a 1% TFA quench. Hydrolysis product (triclosan) was separated from Compound (9) by use of an HP1100 series HPLC equipped with an Alltech Adsorbosphere HS(C₁₈)5μ150 mm×4.6 mm column. The mobile phase was isocratic containing 55% acetonitrile and 0.1% TFA producing retention times of 20.4 minutes and 24.1 minutes for triclosan and Compound (9), respectively. Flow rate was 1 ml⁻¹. Quantitation of triclosan was based on the A260 integrated peak area as compared to triclosan standards.

Bacterial Strains

Escherichia coli BL21 (DE3) and Escherichia coli N (Novablue) were obtained from Novagen (Madison, Wis.). The Escherichia coli/TEM-1 clone was generated by transforming plasmid pcDNA3.1(−) (Invitrogen, San Diego, Calif.), which constitutively express TEM-182 -lactamase, into Escherichia coli N. Bacterial strains used for determining antimicrobial activity included Staphylococcus aureus ATCC 700698, 700699, 43300, 700787, 700788, 700789, 33591, 33592, 33593, 33594, 700260, 13301, 11632, 14154, Staphylococcus epidermidis ATCC27626, 700565, 700566, 700578, 700583, Enterococcus faecalis ATCC 49149, 700802, 49757, 49532, 49533, 51299, 51575, Enterococcus faecium ATCC51559, 700221, 49224, 51558, 49225, 49032, Enterobacter aerogenes ATCC 29757, 29009, 13048, 29007, 35028, Enterobacter cloacae ATCC 23355, Klebsiella pneumoniae ATCC 51503, 700721, 51504, 27799, 15380, Moraxella catarrhalis ATCC 49265, 51584, 43627, 43628. Haemophilus influenzae ATCC 33533, 51584, 43627, 43628, and Pseudomonas aeruginosa ATCC 29872, 21776, 21726, 27853. All above strains were obtained from American Type Culture Collection (ATCC, Manassas, Va.). The 31 recent clinical isolates of Staphylococcus aureus were from Clinical Microbiology laboratory at University of California, San Diego, Medical Center. Streptococcus pneumoniae 1629, N1387 and ERY2 were from Smithkline Beecham (West Sussex, U.K.)

Escherichia coli was grown in LB (Difco) medium. All other strains were grown in the medium recommend by ATCC.

Detection of Triclosan Released from Compound (9) in Escherichia coli/TEM-1

Overnight cultures of both wild-type Escherichia coli N and β-lactamase producing strain Escherichia coli/TEM-1 were diluted 100 times with fresh LB medium and cells were grown for 4 hours at 37° C. (shaking at 230 rpm). Cells were then diluted with fresh LB medium containing 100 μM Compound (9) until the OD₆₀₀ reached 0.2. Aliquots of the culture were sampled at different timed intervals and the cells were pelleted by centrifugation at 10,000×g for 2 minutes at 4° C. The supernatant was removed and combined with 500 μl acetonitrile. The pelleted bacterial cells were resuspended in 100 μl 133 Phosphate Buffer (GIBCO-BRL, pH8.0) and subjected to 3 cycles of freeze-thawing, followed by addition of 500 μl acetonitrile. Both pellet extracts and supernatant were centrifuged at 10,000×g for 2 minutes at 4° C. 550 μl of the resultant supernatant was transferred into a new Eppendorf tube and vacuum-dried. Dried pellets were resuspended in 50 μl H₂O and 100 μl acetonitrile, 75 μl of which was analyzed by HPLC.

Susceptibility Testing

The NCCLS (National Committee for Clinical Laboratory Standards) method to determine MIC's of antimicrobial compounds was modified for high-throughput screening. All stocks of tested compounds were prepared in either water or in dimethylsulfoxide (DMSO), depending on the solubility. At the highest tested concentration, DMSO content did not exceed 0.5%. Briefly 20 2-fold serial dilutions of testing compounds from the highest concentration were made in a 384-well microtiter plate. Each well was inoculated with testing bacteria in broth to a final concentration of approximately 1-1.5×10⁶ cells/ml. Bacterial growth was determined by the increase of optical density at 600 nm using a microplate reader (Tecan SpectraFluor Plus). The MIC was defined as the lowest concentration at which bacteria growth was inhibited after 16-18 hours of incubation at the appropriate temperature required for the bacteria growth. All bacterial cultures were tested for β-lactamase production by use of nitrocefin, following the manufacturer's instruction (Calbiochem, cat. No.484400).

Bactericidal Assays

Overnight broth cultures of S. aureus (ATCC#700260) were diluted 100 times into liquid medium and allowed to grow to exponential phase (OD₆₀₀=0.6). Next the bacteria were diluted into fresh medium to give a working concentration of 10⁶ cells/ml. Compound (9) was added to a final concentration of 0.6 μg/ml, and the suspension was incubated at 37° C. Aliquots of samples were removed at timed intervals of hour 0, 4 and 24 post addition of Compound (9). The samples were diluted at 10⁻³×, 10⁻⁴× or 10⁻⁵× fold, and plated onto agar plates. The plates were incubated for 24 hours at 37° C. to obtain a viable cell count.

Glucuronidation Assay

Glucuronidation assay was performed based on modified procedure from Bansal and Gessner (1980) Anal. Biochem. 109:321-329. Reactions were performed in 50 mM Tris buffer, pH 7.6, containing 10 mM MgCl₂, ImM UDPGA, 0.04 μCi of ¹⁴C-UDPGA, 0.1 mM substrate, and 100 μg human liver microsome. The final volume was 100 μL. Reactions were incubated at 37° C. for one hour and the reactants extracted with 200 μL of 100% ethanol. The protein was removed by centrifugation at 12,000×g for 5 minutes in an Eppendorf centrifuige. The supernatant was dried and resuspended in 30 μL of 100% methanol and spotted on a Whatman glass-backed linear k TLC plate. Chromatography was performed in a mixture of 1-butanol/acetone/acetic acid/water (35:35:10:20). The TLC plate was then dried and exposed to phosphor imager overnight, then monitored by imaging (MolecularDynamics Storm 820).

### Results

Design and Synthesis of Compound (9)

The generic design of β-lactamase ECTA compounds includes a cephalosporin backbone, which incorporates a prodrug form of bactericidal agent at the 3′-position of the β-lactam ring. Hydrolysis by β-lactamase forms and initiates release of the bactericide. Compound (9) was prepared by the condensation of cephalothin with triclosan under Mitsunobu reaction conditions (See U.S. Pat. No. 5,801,242) followed by the deprotection (See synthesis scheme, above).

In vitro TEM-1 Enzyme Assay of Compound (9)

TEM-1 is one of the most common β-lactamases found in clinical isolates. Compound (9) acted as a substrate of this enzyme. As shown in Table 3, Compound (9) possesses comparable reaction characteristics to the commercially available chromogenic substrate nitrocefin.

TABLE 3
A Comparison of the β-lactamase TEM-1 kinetic
constants for Compound (9) and nitrocefin

| Substrate | kcat^{a}(s − 1) | Km^{a}(μM) | kcat/Km(M⁻¹s⁻¹) |
|---|---|---|---|
| Compound (9) | 0.50 | 6.5 | 7.7 × 10⁴ |
| Nitrocefin | 3.3 | 15 | 22 × 10⁴ |

Compound (9) catalysis was determined via fixed time incubations at varied concentrations followed by acid quenching. Product formation (triclosan) was determined by integrated peak area of A260 nm following HPLC separation. Actual triclosan concentrations were determined based on injected triclosan standards.
^{a}Compound (9) kcat and Km were determined from a progress curve analysis fit to the integrated Michaelis-Menton equation at 16 μM Compound (9) and 12.5 nM TEM-1 β-lactamase.

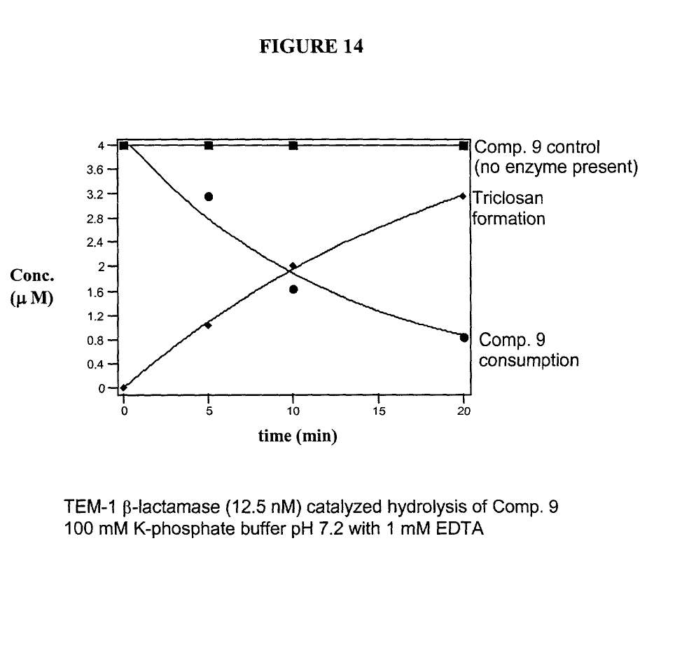

With a Kcat/Km of 21,000±820 M⁻¹ s⁻¹, bacteria expressing TEM-1 β-lactamase in sufficient quantity to resist β-lactam antibiotics were expected to produce bactericidal quantities of triclosan. TEM-1 β-lactamase mediated hydrolysis of Compound (9) resulted in the concomitant production of triclosan. (See FIG. 14). In 20 minutes, 12.5 nM TEM-1 converted almost 90% of a 4 μM solution of Compound (9) to triclosan.

Triclosan Formed from Compound (9) in vivo

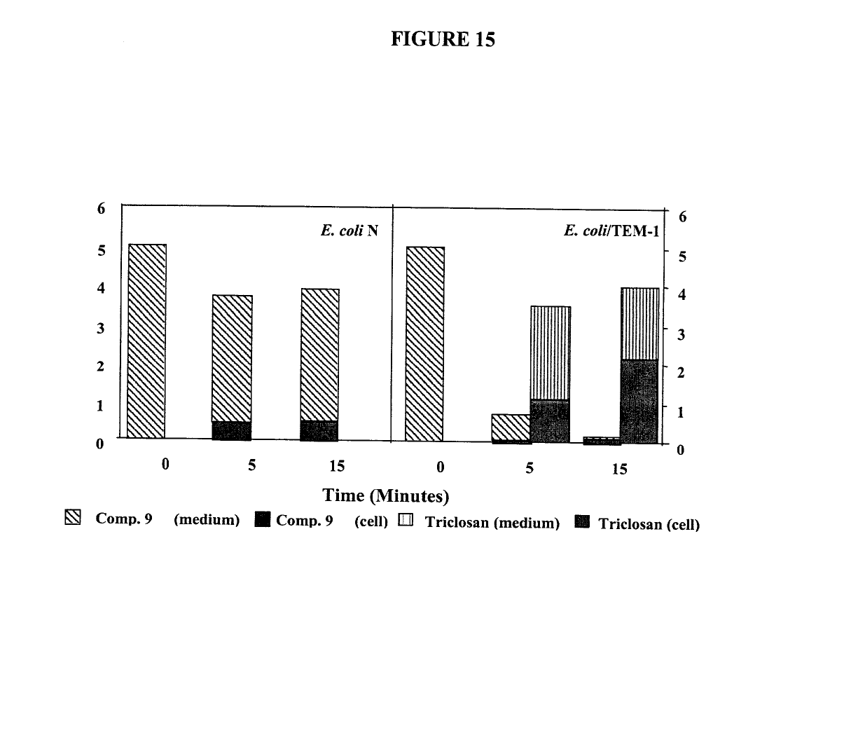

Triclosan formed in vivo was examined in an E. coli model system. The cloned β-lactamase producing strain E. coli/TEM-1 and its parental strain E. coli N, differing only in the expression of TEM-1, were used to characterize the role of β-lactamase on the formation of triclosan from Compound (9). E. coli N and E. coli/TEM-1 were treated with Compound (9), and aliquots of the cultures were sampled at timed intervals. Quantitation of Compound (9) and triclosan was by use of HPLC. As shown in FIG. 15, after five minutes, approximately 80% of Compound (9) was hydrolyzed producing an equimolar amount of triclosan in the E. coli/TEM-1 sample.

On the other hand, triclosan formation was below the limit of detection in E. coli N, up to 15 minutes post-treatment. These results demonstrate the importance role of β-lactamase in production of triclosan from Compound (9).

The Antibacterial Activity of Compound (9) in an Escherichia coli Model System.

The in vivo β-lactamase dependence of Compound (9) was initially demonstrated in the E. coli model system. As shown in Table 4, Compound (9) was 128 fold more potent against the β-lactamase producing strain E. coli/TEM-1 compared with the β-lactamase negative strain E. coli N.

TABLE 4
Activity of Compound (9) with or without β-lactamase inhibitors against
wild-type and β-lactamase producing E. coli/TEM-1

| | | | MIC (μg/mL) | | |
|---|---|---|---|---|---|
| | | | | Comp. (9) plus | |
| Cells | Cephalothin | Phenotype | Comp. (9) | 4 μg/mL CLA | 8 μg/mL SBT |
| E. coli | BLA neg. | 0.25 | 0.25 | 0.13 | 16 |
| E. coli/TEM-1 | BLA pos. | 0.002 | 0.25 | 0.016 | >32 |

β-lactamase expressing strain E. coli/TEM-1 was cloned from β-lactamase negative E. coli N transformed with TEM-1. The MICs were determined according to NCCLS standards.
BLA, β-lactamase.
CLA, Lithium clavulanate, CLAV alone has no effect on the tested cells at concentration of 4 μg/mL.
SBT, Sulbactam, SBT alone has no effect on the tested cells at concentration of 8 μg/mL.

As a further demonstration of the ECTA dependence of Compound (9), the enhanced sensitivity of E. coli/TEM-1 to Compound (9) was diminished by the addition of β-lactamase inhibitors such as clavulanate and sulbactam. These results demonstrate that the enhanced antibacterial activity of Compound (9) on E. coli/TEM-1 is β-lactamase-dependent. In other words, production of β-lactamase in bacteria strain leads to enhanced sensitivity to Compound (9).

In vitro Activity of Compound (9) Against Staphylococcus aureus

Most drug resistant isolates of S. aureus produce β-lactamase (See Medeiros (1984) Br. Med. Bull. 40:18-27; Maranan (1997) Infect. Dis. Clin. North Am. 11:813-849). The in vitro antibacterial activity of Compound (9) in S. aureus was compared with a panel of reference antibiotics, including the antibiotics of choice (AOC). Overall, Compound (9) exhibited excellent activity against 14 tested strains of S. aureus (geometric mean MIC was 0.001 μg/ml) (See Table 5A below).

TABLE 5A
Antibacterial activities of Compound (9) and reference antibiotics against
Staphylococcus aureus.

| | | MIC (μg/mL) | | | |
|---|---|---|---|---|---|
| | β-lactamase | | Vanco- | | |
| ATCC # | (Nitrocefin) | Comp. (9) | mycin | Cephalothin | Oxacillin |

Methicillin-resistant:

| | | | | | |
|---|---|---|---|---|---|
| 700699 | Negative | 2 | 4 | 100 | >32 |
| 700698 | Negative | 1 | 2 | 100 | >32 |
| 700787 | Positive | 1 | 8 | 50 | >32 |
| 700788 | Positive | 0.25 | 2 | 3.13 | >32 |
| 700789 | Positive | 0.031 | 2 | 6.25 | 32 |
| 33591 | Positive | 0.00003 | 0.25 | .56 | 8 |
| 33593 | Positive | 0.000015 | 0.5 | 0.78 | 8 |

Methicillin-sensitive:

| | | | | | |
|---|---|---|---|---|---|
| 43300 | Neg./Pos. | 0.00006 | 0.5 | 0.39 | 2 |
| 33592 | Positive | 0.000015 | 0.5 | 1.56 | 2 |
| 14154 | Positive | 0.000015 | 0.5 | 0.39 | 0.5 |
| 700260 | Positive | 0.000015 | 1 | 0.39 | 0.5 |
| 33594 | Positive | 0.000015 | 0.5 | 0.2 | 0.25 |
| 13301 | Positive | 0.000015 | 0.25 | 6.25 | 0.25 |
| 11632 | Neg./Pos. | 0.000015 | 0.25 | 0.2 | 0.25 |

All tested bacterium were obtained from ATCC. MICs were determined based upon NCCLS (modified for high-through-put screening). Methicillin resistance was determined with MIC of oxacillin 4 μg/mL Producing of β-lactamase was tested with nitrocefin.

Furthermore, the data for the antibacterial activity of Compound (9) and the reference compounds towards all tested S. aureus was summarized as the groups of β-lactamse-producing or non-producing strains. as well as the groups of methicillin-resistant or nonresistant strains. As shown in Table 5B, Compound (9) exhibited higher activity against β-lactamnase-producing than non-producing S. aureus.

TABLE 5B
Summary of Compound (9)'s Activity Against Staphylococcus Aureus

| | Geometric mean of MIC (μg/ml) | | | |
|---|---|---|---|---|
| Organisms | Comp. 9 | | | |
| (# of tested isolates) | Oxacillin | Vancomycin | Cephalothin | |
| S. aureus (14) | 0.001 | 0.86 | 2.45 | |
| >3.45 | | | | |
| Based upon β- | | | | |
| lactamase-producing | | | | |
| β-lactamase | 1.41 | 2.83 | 100 | >32 |
| negative (2) | | | | |
| β-lactamase | 0.00019 | 0.94 | 1.32 | |
| positive (12) | | | | |
| 2.34 | | | | |
| Based upon resistant | | | | |
| to methicillin | | | | |
| MRSA tested (7) | 0.025 | 1.64 | 10.25 | |
| >21.5 | | | | |
| MSSA tested (7) | 0.000018 | 0.36 | 0.58 | |
| 0.55 | | | | |

All tested bacterium were obtained from ATCC. MICs were determined based upon NCCLS (modified for high-through-put screening). Methicillin resistance was determined with MIC of oxacillin 4 μg/mL. Production of β-lactamase was tested with nitrocefin.

The geometric mean MIC for β-lactamase-producing strains (0.00019 μg/ml) was substantially lower than that of β-lactamase non-producing (1.57 μg/mL). Again, this result supports the concept of using β-lactamase as an engine for the activation of ECTA compounds in S. aureus.

Vancomycin has been considered the last resort for the treatment of the infectious diseases caused by methicillin resistant S. aureus (MRSA) (See Medeiros (1984) Br. Med. Bull. 40:18-27). Currently, there are no effective antibiotics against vancomycin-resistant S. aureus in clinical use. Compound (9) was tested against both MRSA and vancomycin-resistant S. aureus and displayed at least 85-fold greater potency than vancomycin (See Table SB above).

Considering the possible variability between ATCC's strains and more recent clinical isolates, 14 strains of methicillin sensitive S. aureus (MSSA) (MIC was <5 μg/ml with cefazolin) and 17 strains of MRSA (MIC was 5 μg/ml with cefazolin) from the clinical microbiology laboratory at University of California, San Diego, Medical Center were obtained. Growth of all 14 isolates of MSSA was inhibited by Compound (9) at the lowest tested concentration (0.08 μg/ml) (See Table 6, below). In addition, Compound (9) is also highly active (MIC₅₀ is 0.08 μg/ml and MIC₉₀ is 0.3 μg/ml) against MRSA (See Table 5).

TABLE 6
Summarized Activity of Compound (9) Against Most Recent Clinical
Isolated S. aureus (Tested in Clinical Microbiology Laboratory
at University of California, San Diego, Medical Center)

| Organism | | MIC range | MIC₅₀ | MIC₉₀ |
|---|---|---|---|---|
| (no. of strains) | Drug | (μg/ml) | (μg/ml) | (μg/ml) |
| MSSA (14) | Comp. (9) | 0.08-0.08 | 0.08 | 0.08 |
| | Cefazolin | 0.08-0.6 | 0.16 | 0.3 |
| MRSA (17) | Comp. (9) | 0.08-0.6 | 0.08 | 0.3 |
| | Cefazolin | 5 | >5 | >5 |

All tested S. aureus were obtained from ATCC. MICs were determined by agar dilution method, which was used for routine test in Clinical Microbiology Lab, Medical Center, University of California at San Diego. Methicillin resistance was determined with MIC of cefazolin 5 μg/mL.

In vitro Antibacterial Activity of Compound (9) Against Other Pathogens

The antibacterial activity of Compound (9) was evaluated against a number of common pathogens including Gram-positive strains such as S. epidermidis, E. faecalis, E. faecium, S. pneumoniae and Gram-negative strains such as M. catarrhalis, H. influenzae, E. aerogenes, E. cloacae, K. pneumoniae.

Compound (9) was 435-fold more active than vancomycin against S. epidermidis with a geometric mean MIC of 0.004 μg/ml. More significantly, 4 of the 5 tested strains were heterogeneous vancomycin resistant (MICs were 2 μg/ml, See Table 7).

TABLE 7
Summarized antibacterial activities of Compound
(9) and reference antibiotics against gram-positive
(other than S. aureus) clinical isolates

| Organism(s) | | MIC range | GM* MIC |
|---|---|---|---|
| (no. of strains) | Drug | (μg/ml) | (μg/ml) |
| Gram-positive strains: | | | |
| S. epidermidis (5) | Comp. (9) | 0.000015-0.5 | <0.004 |
| | Vancomycin | 1-2 | 1.74 |
| | Oxacillin | 0.69->11 | >7.87 |
| | Cephalothin | 0.2-25 | 1.81 |
| S. pneumoniae (3) | Comp. (9) | 0.06-0.5 | 0.122 |
| | Cephalothin | 0.06-2 | 0.247 |
| E. faecalis (7) | Comp. (9) | 8-16 | 8 |
| | Vancomycin | 1->125 | >6.41 |
| | Oxacillin | >11 | >11 |
| | Cephalothin | 25-100 | 60.95 |
| E. faecium (6) | Comp. (9) | 2->16 | >7 |
| | Vancomycin | 1->125 | >3.49 |
| | Cephalothin | >100 | >100 |
| Including VRE **(5) | Comp. (9) | 8-16 | 10.3 |
| | Vancomycin | 16-> 125 | >47.4 |

All tested bacterium were obtained from ATCC. MICs were determined modified NCCLS method.
*Geometric mean
**Vancomycin-resistant E. faecalis or E. faecium.

Towards vancomycin-resistant E. faecalis or E. faecium, Compound (9) also exhibited significant high activity: the geometric mean MIC was 10.3 μg/ml, whereas the geometric mean MIC of vancomycin on the same strains was >47.4 μg/ml. However, the activities of Compound (9) towards E. faecalis and E. faecium, which are susceptible to vancomycin, were comparable to that of vancomycin. These results demonstrated the potential of Compound (9) as an effective antibiotic against clinically significant vancomycin resistant species. In another clinically important strain responsible for community acquired pneumonia, S. pneumoniae sensitivity to Compound (9) was also observed.

Compound (9) is also extremely active against Gram-negative species. The geometric mean MIC of ±0.00018 μg/ml for M. catarrhalis and 0.015 μg/ml for H. influenzae were at least 1000 fold stronger than those of ampicillin and cephalothin. Against E. aerogenes, E. cloacae and K. pneumoniae, Compound (9) was at least 33 fold more active than cephalothin or ampicillin, and only 4 fold less active than imipenem. However, Compound (9) showed no activity against P. aeruginosa (See Table 8).

TABLE 8
Summarized Antibacterial Activities of Compound (9) and Reference
Antibiotics Against Gram-Negative Clinical Isolates.

| Organism(s) | Drug | | GM* MIC |
|---|---|---|---|
| (no. of strains) | (μg/ml) | MIC range | (μg/ml) |
| M. catarrhalis (4) | Comp. (9) | 0.000015-0.031 | 0.00018 |
| H. influenzae (4) | Comp. (9) | 0.0075-0.061 | 0.015 |
| E. cloacae (1) | Comp. (9) | 1 | 1 |
| | Ampicillin | 31 | 31 |
| | Imipenem | 0.0078 | 0.0078 |
| | Cephalothin | >100 | >100 |
| E. aerogenes (5) | Comp. (9) | 0.5-8 | 3.36 |
| | Ampicillin | 61-7813 | 488 |
| | Imipenem | 0.5-1 | 0.66 |
| | Cephalothin | >100 | >100 |
| K. pneumoniae (5) | Comp. (9) | 0.98-3.91 | 1.96 |
| | Ampicillin | 61-125,000 | 27,201 |
| | Imipenem | 0.063-8 | 0.46 |
| | Cephalothin | 12.5->100 | >66 |
| P. aeruginosa (7) | Comp. (9) | >15.6 | >15.6 |

All tested bacterium were obtained from ATCC. MICs were determined based upon NCCLS (modified for high-through-put screening).

Bactericidal Activity of Compound (9)

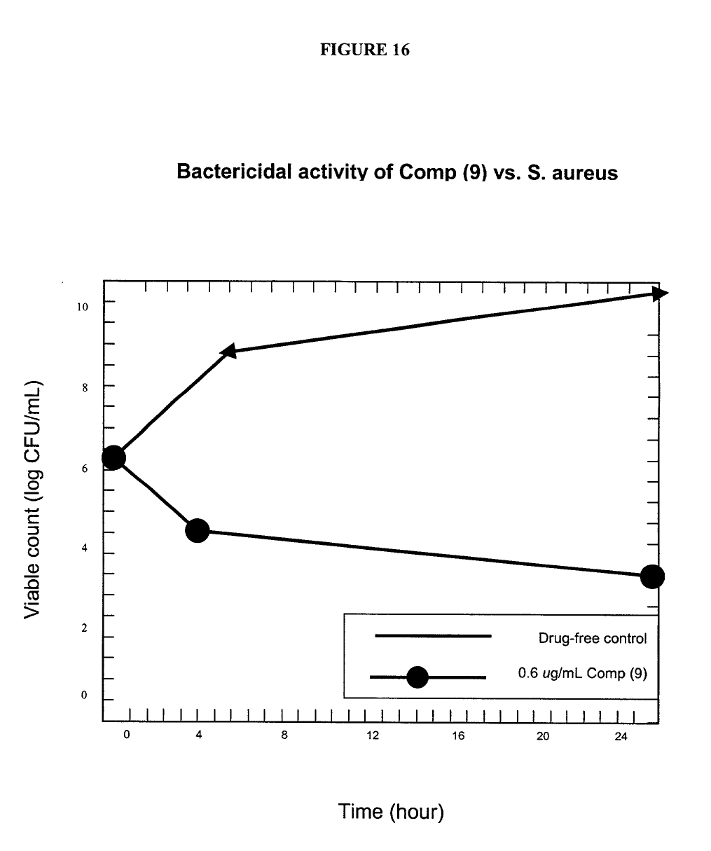

The bactericidal effect of Compound (9) was evaluated using S. aureus, (ATCC #700260). At a concentration of 0.6 μg/ml, Compound (9) showed cidal activity by decreasing the number of viable cells by 100-fold during a 6 hours incubation. After 24 hour incubation, the number of viable cells decreased by 10,000-fold as compared with the cell number at time 0 (See FIG. 16). These data prove that Compound (9) has strong bactericidal activity.

Glucuronidation Assay of Compound (9)

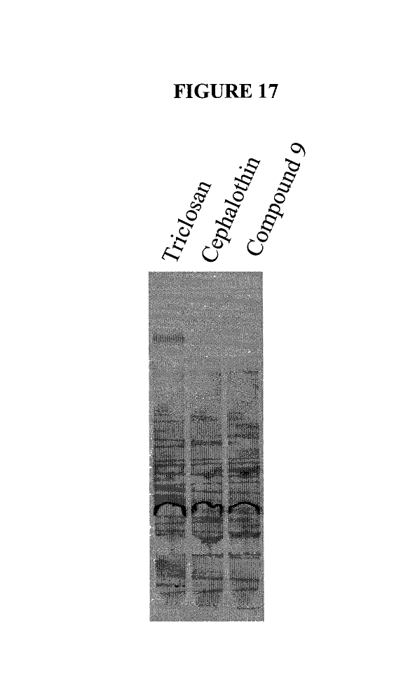

Compound (9), triclosan or cephalothin was incubated with human liver microsome and the possible glucuronides were analyzed by TLC plate. Results (See FIG. 17) indicate that Compound (9) is not a substrate of UDP-glucuronosyltransferase, which is one of the major enzymes of detoxification. triclosan was glucuronidated.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

We claim:

1. A compound of the following structure: 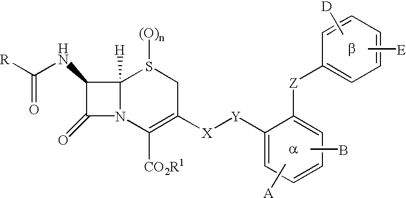
wherein n is 0, 1, or 2;
wherein A, B, D, and E are independently the same, different or absent and are selected from the group consisting of a halogen, H. CN, NO₂, CF₃, C(O)H, NH₂, N(R²)ₙ₁, and C(O)CH₃, OR², wherein R² is selected from the group consisting of H, lower alkyl, alkenyl group, and alkynyl group and wherein n1 is 0, 1 or 2;
wherein X is selected from the group consisting of CH₂, cis-CH═CH—CH₂—, trans-CH═CH—CH₂, —CH₂—O—C(O)—, —NH—C(O)—O—, —C≡C—CH₂, —PO₃—, —SO₃—, —SO₂, —NH—CH₂—CH₂—CH₂—NH—CO—, traceless Linker, and 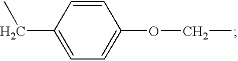
wherein Y is selected from the group consisting of —O—, —S—, and NR³, wherein R³ is selected from the group consisting of H, lower alkyl, alkenyl group, and alkynyl group;
wherein Z is selected from the group consisting of —O—, —C(O)—, —S—, α-C(O)—N(R⁴)-β, α-N(R⁴)—C(O)-β, and N(R⁴)ₙ₂, wherein R⁴ is selected from the group consisting of H, OH, R⁵, and OR⁵, wherein R⁵ is selected from the group consisting of H, lower alkyl, alkenyl group, and alkynyl group and wherein n2 is 0, 1 or 2;
wherein ring α connects Y to Z and is a benzene or a heterocycle selected from the group consisting of 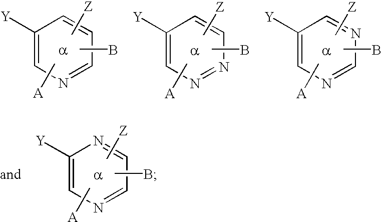
wherein ring β connects to Z and is a benzene or a heterocycle selected from the group consisting of 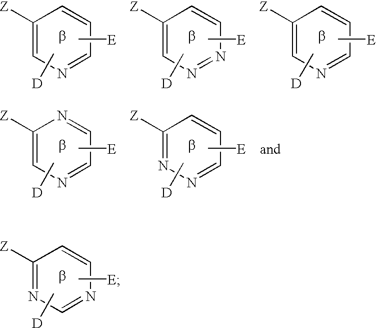
wherein R is selected from the group consisting of Ph-, PhCH₂— and PhOCH₂; or a structure selected from: 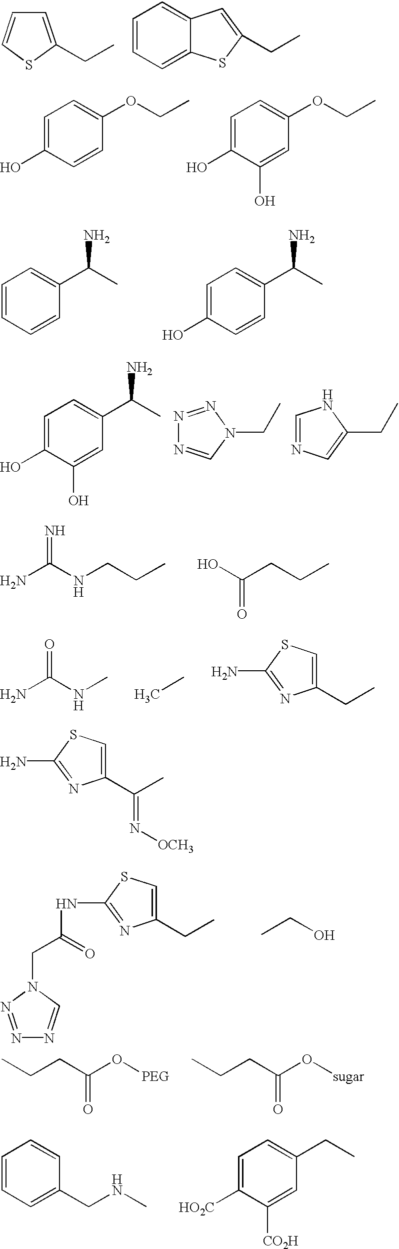
wherein R¹ is selected from the group consisting of H, Li, Na, sugar, THAM (2-amino-2-hydroxymethyl-1,3-propanediol), ammonium, methylamine, dimethylamine, lower alkylamine, bis(lower alkyl)amine and polyethylene glycol (PEG); and derivatives and pharmaceutically acceptable salts of the compounds.

2. The compound of claim 1, wherein n is 0 or 1.

3. The compound of claim 2, wherein X is —CH₂—.

4. The compound of claim 3, wherein Y is O.

5. The compound of claim 4, wherein Z is O.

6. The compound of claim 5, wherein the compound has the following structure:

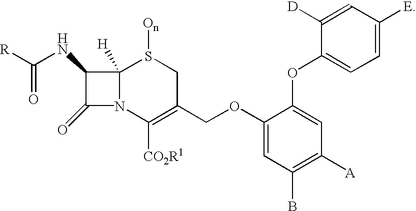

7. The compound of claim 6, wherein B, D and E are each halogens.

8. The compound of claim 7, wherein B, D and F are Cl.

9. The compound of claim 8, wherein A is hydrogen.

10. The compound of claim 9, wherein R is selected from the group consisting of

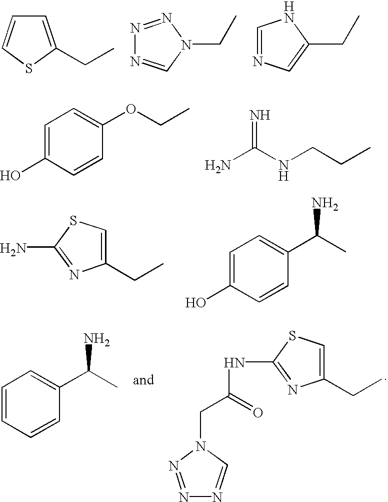

11. The compound of claim 10, wherein R¹ is hydrogen.

12. The compound of claim 11, wherein n is 0.

13. The compound of claim 12, wherein the compound is 3-(2-(2,4-dichlorophenoxy)-5-chlorophenoxy)methyl-7-(2-thienylacetamido)-3-cephem-4-carboxylic acid (Compound 9).

14.The compound of claim 12, wherein the compound is 3-(2-(2,4-dichlorophenoxy)-5-chlorophenoxy)methyl-7-(1-tetrazoleacetamido)-3-cephem-4-carboxylic acid (Compound 29).

15. The compound of claim 12, wherein the compound is 3-(2-(2,4-dichlorophenoxy)-5-chlorophenoxy)methyl-7-[2-(3H-imidazol-4-yl)]-acetamido-3-cephem-4-carboxylic acid (Compound 31).

16. The compound of claim 12, wherein the compound is 3-(2-(2,4-dichlorophenoxy)-5-chlorophenoxy)methyl-7-(1-phenyl-2-aminoacetamido)-3-cephem-4-carboxylic acid (Compound 38).

17. The compound of claim 12, wherein the compound is 3-(2-(2,4-dichlorophenoxy)-5-chlorophenoxy)methyl-7-[4-(2-aminothiazole)-yl-2-acetamido]-3-cephem-4-carboxylic acid (Compound 39).

18. The compound of claim 12, wherein the compound is 3-(2-(2,4-dichlorophenoxy)-5-chlorophenoxy)methyl-7-[2-(4-hydroxyphenoxy)acetamido]-3-cephem-4-carboxylic acid (Compound 40).

19. The compound of claim 12, wherein the compound is 3-(2-(2,4-dichlorophenoxy)-5-chlorophenoxy)methyl-7-[2-amino-2-(4-hydroxy-phenyl)acetamido)-3-cephem-4-carboxylic acid (Compound 41).

20. The compound of claim 12, wherein the compound is 3-(2-(2,4-dichlorophenoxy)-5-chlorophenoxy)methyl-7-(3-guanidinopropyl)acetamido-3-cephem-4-carboxylic acid (Compound 42).

21. The compound of claim 12, wherein the compound is 3-[5-chloro-2-(2,4-dichlorophenoxy)-phenoxymethyl]-7-{2-[2-(2-tetrazol-1-yl-acetamido)-thiazol-5-yl]-acetamido-3-cephem-4-carboxylic acid (Compound 43).

22. The compound of claim 11, wherein n is 1.

23. The compound of claim 22, wherein the compound is 3-(2-(2,4-dichlorophenoxy)-5-chlorophenoxy)methyl-7-(2-thienylacetamido)-1-oxo-3-cephem-4-carboxylic acid (Compound 11).

24. The compound of claim 10, wherein R¹ is polyethylene glycol (PEG).

25. The compound of claim 24, wherein n is 0.

26. The compound of claim 25, wherein the compound has the following structure: 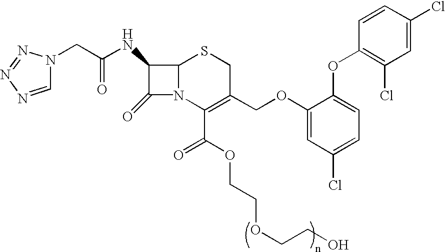
wherein n is 4 to 2000. (Compound 32)

27. The compound of claim 2, wherein X is cis-CH═CH—CH₂— or trans-CH═CH—CH₂.

28. The compound of claim 27, wherein Y is O.

29. The compound of claim 28, wherein Z is α-C(O)—N(R⁴)-β.

30. The compound of claim 29, wherein R⁴ is hydrogen.

31. The compound of claim 30, wherein the compound has the following structure:

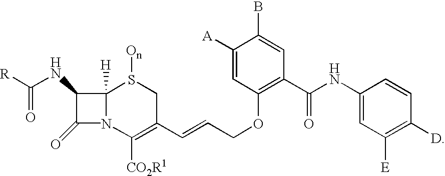

32. The compound of claim 31, wherein R¹ is hydrogen.

33. The compound of claim 32, wherein R is

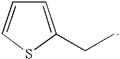

34. The compound of claim 33, wherein B, D, and E each halogens.

35. The compound of claim 34, wherein B, D and E are Cl.

36. The compound of claim 35, wherein A is hydrogen.

37. The compound of claim 36, wherein n is 0.

38. The compound of claim 37, wherein the compound is 3-{3-[4-chloro-2-(3,4-dichloro-phenylcarbamoyl)-phenoxy]-propenyl}-7-(2-thiophene-acetmido)-3-cephem-4-carboxylic acid (Compound 35).

39. A composition comprising the compounds of claim 1 and a carrier.

40. The composition of claim 39, wherein the carrier is a pharmaceutically acceptable carrier.

41. A method of inhibiting the growth of a microorganism comprising contacting the microorganism with an effective amount of the compound of claim 1.

42. The method of claim 41, wherein the microorganism expresses β-lactamase.

43. The method of claim 42, wherein the microorganism is selected from the group consisting of Staphylococcus aureus, Staphylococcus epidermidis and other coagulase-negative staphylococci, Streptococcus pyogenes, Streptococcus pneumoniae, Streptococcus agalactiae, Enterococcus species, Corynebacterium diphtheriae, Listeria monocytogenes, Bacillus anthracis, Neisseria meningitidis, Neisseria gonorrhoeae, Moraxella catarrhalis, Vibrio cholerae, Campylobacter jejuni, Enterobacteriaceae (includes: Escherichia, Salmonella, Klebsiella, Enterobacter), Pseudomonas aeruginosa, Acinetobacter species, Haemophilus influenzae, Clostridium tetani, Clostridium botulinum, Bacteroides species, Prevotella species, Porphyromonas species, Fusobacterium species, Mycobacterium tuberculosis, and Mycobacterium leprae,
with the proviso that when the compound is 3-(2-(2,4-dichlorophenoxy)-5-chlorophenoxy)methyl-7-(2-thienylacetamido)-3-cephem-4-carboxylic acid, the microorganism is not Pseudomonas aeruginosa.

44. The method of claim 41, wherein the microorganism is vancomycin resistant, tolerant or sensitive.

45. The method of claim 44, wherein the vancomycin resistant, tolerant or sensitive microorganism is selected from the group consisting of Staphylococcus aureus, Staphylococcus epidermis, Enterococcus faecalis and Enterococcus faecium.

46. A method for inhibiting penicillin binding protein in an infected cell comprising contacting the cell with an effective amount of claim 1.

47. The method of claim 46, wherein the infected cell is vancomycin resistant, tolerant or sensitive.

48. The method of claim 47, wherein the vancomycin resistant, tolerant or sensitive infected cell is selected from the group consisting of Staphylococcus aureus, Staphylococcus epidermis, Enterococcus faecalis and Enterococcus faecium.

49. A method for treating a subject infected with a microorganism, comprising administering to the subject an effective amount of the compound of claim 1, thereby treating the subject.

50. The method of claim 49, wherein the microorganism produces -lactamase.

51. The method of claim 50, wherein the microorganism is selected from the group consisting of Staphylococcus aureus, Staphylococcus epidermidis and other coagulase-negative staphylococci, Streptococcus pyogenes, Streptococcus pneumoniae, Streptococcus agalactiae, Enterococcus species, Corynebacterium dipahtheriae, Listeria monocytogenes, Bacillus anthracis, Neisseria meningitidis, Neisseria gonorrhoeae, Moraxella catarrhalis, Vibrio cholerae, Campylobacter jejuni, Enterobacteriaceae (includes: Escherichia, Salmonella, Klebsiella, Enterobacter), Pseudomonas aeruginosa, Acinetobacter species, Haemophilus influenzae, Clostridium tetani, Clostridium botulinum, Bacteroides species, Prevotella species, Porphyromonas species, Fusobacterium species, Mycobacterium tuberculosis, and Mycobacterium leprae,
with the proviso that when the compound is 3-(2-(2,4-dichlorophenoxy)-5-chlorophenoxy)methyl-7-(2-thienylacetamido)-3-cephem-4-carboxylic acid, the microorganism is not Pseudomonas aeruginosa.

52. The method of claim 49, wherein the microorganism is vancomycin resistant, tolerant or sensitive.

53. The method of claim 52, wherein the vancomycin resistant, tolerant or sensitive microorganism is selected from the group consisting of Staphylococcus aureus, Staphylococcus epidermis, Enterococcus faecalis and Enterococcus faecium.

54. A method of screening for an antibacterial agent comprising contacting a sample containing a bacterial cell with a test agent and contacting a second sample containing the bacterial cell with a compound of claim 1 and comparing the ability of each to inhibit the growth of the bacterial cell.

55. The method of claim 54, wherein the bacterial cell produces β-lactamase.

56. The method of claim 55, wherein the bacterial cell is selected from the group consisting of Staphylococcus aureus, Staphylococcus epidermidis and other coagulase-negative staphylococci, Streptococcus pyogenes, Streptococcus pneumoniae, Streptococcus agalactiae, Enterococcus species, Corynebacterium diphtheriae, Listeria monocytogenes, Bacillus anthracis, Neisseria meningitidis, Neisseria gonorrhoeae, Moraxella catarrhalis, Vibrio cholerae, Campylobacter jejuni, Enterobacteriaceae (includes: Escherichia, Salmonella, Klebsiella, Enterobacter), Pseudomonas aeruginosa, Acinetobacter species, Haemophilus influenzae, Clostridium tetani, Clostridium botulinum, Bacteroides species, Prevotella species, Porphyromonas species, Fusobacterium species, Mycobacterium tuberculosis, and Mycobacterium leprae,
with the proviso that when the compound is 3-(2-(2,4-dichlorophenoxy)-5-chlorophenoxy)methyl-7-(2-thienylacetamido)-3-cephem-4-carboxylic acid, the bacteria is not Pseudomonas aeruginosa.

57. The method of claim 54, wherein the bacterial cell is vancomycin resistant, tolerant or sensitive.

58. The method of claim 57, wherein the vancomycin resistant, tolerant or sensitive bacterial cell is selected from the group consisting of Staphylococcus aureus, Staphylococcus epidermis, Enterococcus faecalis and Enterococcus faecium.

59. A compound of the following structure: 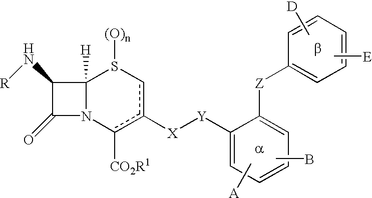
wherein n is 0 or 1;
wherein A, B, D, and F are independently the same, different or absent and are selected from the group consisting of a halogen, H, and NO₂;
wherein X is selected from the group consisting of CH₂, cis-CH═CH—CH₂—, trans-CH═CH—CH₂, and —CH₂—O—C(O)—;
wherein Y is —O—;
wherein Z is —O— or α-C(O)—N(H)-β or absent;
wherein ring α connects Y to Z and is a benzene;
wherein ring β connects to Z and is a benzene or is absent;
wherein R is selected from the group consisting of 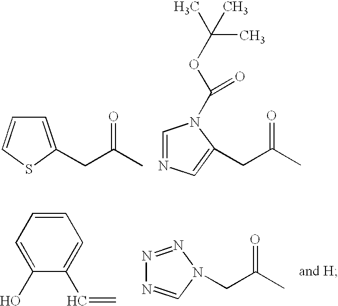
and wherein R¹ is diphenylmethyl or 4-nitrobenzyl.

60. The compound of claim 59, wherein the compound is diphenylmethyl 3-(2-(2,4-dichlorophenoxy)-5-chlorophenoxy)methyl-7-(2-thienylacetamido)-3-cephem-4-carboxylate (Compound 7).

61. The compound of claim 59, wherein the compound is diphenylmethyl 3-(2-(2,4-dichlorophenoxy)-5-chlorophenoxy)methyl-7-(2-thienylacetamido)-2-cephem-4-carboxylate (Compound 8).

62. The compound of claim 59, wherein the compound is diphenylmethyl 3-(2-(2,4-dichlorophenoxy)-5-chlorophenoxy)methyl-7-(2-thienylacetamido)-1-oxo-3-cephem-4-carboxylate (Compound 10).

63. The compound of claim 59, wherein the compound is diphenylmethyl 3-(4-nitrophenoxycarbonyloxy)methyl-7-(2-thienylacetamido)-3-cephem-4-carboxylate (Compound 12).

64. The compound of claim 59, wherein the compound is diphenylmethyl 3-((2-(2,4-dichlorophenoxy)-5-chlorophenoxy)carbonyloxy)methyl-7-(2-thienylacetamido)-2-cephem-4-carboxylate (Compound 14).

65. The compound of claim 59, wherein the compound is diphenylmethyl 3-((2-(2,4-dichlorophenoxy)-5-chlorophenoxy)carbonyloxy)methyl-7-(2-thienylacetamido)-3-cephem-4-carboxylate (Compound 13).

66. The compound of claim 59, wherein the compound is diphenylmethyl 3-((2-(2,4-dichlorophenoxy)-5-chlorophenoxy)carbonyloxy)methyl-7-(2-thienylacetamido)-1-oxo-3-cephem-4-carboxylate (Compound 16).

67. The compound of claim 59, wherein the compound is 4-nitrobenzyl 3-(1-(2-(2,4-dichlorophenoxy)-5-chlorophenoxy)-3-propenyl)-7-(2-thienylacetamido)-3-cephem-4-carboxylate (Compound 23).

68. The compound of claim 59, wherein the compound is diphenylmethyl 3-(2-(2,4-dichlorophenoxy)-5-chlorophenoxy)methyl-7-β-(o-hydroxy)benzylidenamino-3-cephem-4-carboxylate (Compound 26).

69. The compound of claim 59, wherein the compound is diphenylmethyl-3-(2-(2,4-dichlorophenoxy)-5-chlorophenoxy)methyl-7-amino-3-cephem-4-carboxylate (Compound 27).

70. The compound of claim 59, wherein the compound is diphenylmethyl 3-(2-(2,4-dichlorophenoxy)-5-chlorophenoxy)methyl-7-(1-tetrazoleacetamido)-3-cephem-4-carboxylate (Compound 28).

71. The compound of claim 59, wherein the compound is diphenylmethyl 3-(2-(2,4-dichlorophenoxy)-5-chlorophenoxy)methyl-7-[2-(3-tert-butoxycarbonyl-3H-imidazol-4-yl)]-acetamido-3-cephem-4-carboxylate (Compound 30).

72. The compound of claim 59, wherein the compound is diphenylmethyl-3-{3-[4-chloro-2-(3,4-dichloro-phenylcarbamoyl)-phenoxy]-propenyl}-7-(2-thiophene-acetamido)-3-cephem-4-carboxylate (Compound 34).

73. A process for preparing diphenylmethyl 3-(2-(2,4-dichlorophenoxy)-5-chlorophenoxy)methyl-7-β-(o-hydroxy)benzylidenamino -3-cephem-4-carboxylate which comprises reacting an effective amount of a compound having the structure: 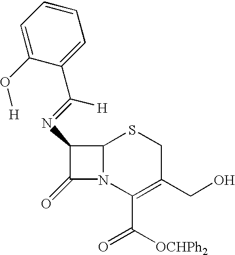
a) with an effective amount of triclosan and cooling;
b) adding an effective amount of triphenylphosphine;
c) adding an effective amount of diisopropylazodicarboxylate and combining with an aqueous solvent and extracting with ethylacetate; and
d) concentrating the compound, thereby preparing diphenylmethyl 3-(2-(2,4-dichlorophenoxy)-5-chlorophenoxy)methyl-7-β-(o-hydroxy)benzylidenamino -3-cephem-4-carboxylate.
